(19) United States
(12) Patent Application Publication
(10) Pub. No.: US 2019/0269700 A1
(43) Pub. Date: Sep. 5, 2019

(54) PHARMACEUTICAL COMPOSITION AND METHODS

(71) Applicant: Lipocine Inc., Salt Lake City, UT (US)

(72) Inventors: Mahesh Patel, Salt Lake City, UT (US); Nachiappan Chidambaram, Sandy, UT (US); Satish Nachaegari, Salt Lake City, UT (US); Srinivasan Venkateshwaran, Salt Lake City, UT (US); Joel Frank, Sandy, UT (US); Chandrashekar Giliyar, Plymouth, MN (US)

(73) Assignee: Lipocine Inc., Salt Lake City, UT (US)

(21) Appl. No.: 16/114,135

(22) Filed: Aug. 27, 2018

Related U.S. Application Data
(63) Continuation of application No. 15/475,070, filed on Mar. 30, 2017, now abandoned.
Application No. 15/475,070 is a continuation of application No. 14/839,564, filed on Aug. 28, 2015, now abandoned.
(60) Provisional application No. 62/043,349, filed on Aug. 28, 2014.

(51) Int. Cl. A61K 31/575; A61P 15/08; A61K 9/48; A61K 31/568; A61K 47/44; A61K 9/00; A61K 47/12; A61K 47/10; A61K 47/14

(52) U.S. Cl. CPC A61K 31/575; A61P 15/08; A61K 9/4858; A61K 31/568; A61K 47/14; A61K 9/0053; A61K 47/12; A61K 47/10; A61K 9/4825; A61K 47/44

(57) ABSTRACT

Disclosed herein are compositions having a lipophilic active agent and methods of their use.

### CROSS-REFERENCE TO RELATED APPLICATIONS

This application is a continuation of U.S. patent application Ser. No. 15/475,070, filed Mar. 30, 2017, which is a continuation of U.S. patent application Ser. No. 14/839,564, filed Aug. 28, 2015, which claims the benefit of U.S. Provisional Application No. 62/043,349 filed Aug. 28, 2014, both of which are incorporated herein by reference in their entirety.

### BACKGROUND OF THE INVENTION

A particularly difficult task facing the pharmaceutical industry is the development of unit dosage forms for oral administration that provide adequate bioavailability of hydrophobic pharmaceutical agents. One difficulty in developing such oral unit dosage forms for hydrophobic drugs is related to the hydrophobicity of the drug and the fact that the oral route of administration is mediated or via an aqueous milieu (e.g., water-based). By definition, hydrophobic drugs have little or no solubility in water and as such, developing a unit dosage form that facilitates the transit of the active agent into (e.g., systemic or lymphatic) circulation can be challenging. Typically, if a drug cannot get into circulation, it cannot exert its therapeutic effect.

To have oral activity, most approved drugs have properties that fall into a particular set of drug-like qualities. The well-known Lipinski's rule of 5, which was based on the evaluation of the physio-chemical properties of more than 2000 drugs and candidates in clinical trials, is used by medicinal chemists to estimate drug-likeness (Lipinski C A, Lombardo F, Dominy B W, Feeney P J (March 2001). Adv. Drug Deliv. Rev. 46 (1-3): 3-26). The rules were based on a 90 percentile of the drug property distribution. One key parameter for a drug-like molecule to have oral biovailability is its log P value (partition coefficient between water and 1-octanol) which Lipinski's rule of 5 says should be lower than 5. Further refinements of this prediction suggest that a log p of between 2 and 3 is optimal for ensuring oral bioavailability (See Thomas et al. Expert Opin. Drug Metab. Toxicol. (2006) 2(4)).

DrugBank is a database (physio-chemical properties and other information) of drugs having 7685 drug entries including 1549 FDA-approved small molecule drugs, 155 FDA-approved biotech (protein/peptide) drugs, 89 nutraceuticals and over 6000 experimental drugs. A search of the set of drugs in DrugBank having a predicted log P of greater than 5 gave 453 hits (for approved drugs 137); greater than 6 gave 187 hits; greater than 7 gave 93 hits (for approved drugs 29); greater than 8 gave 93 hits (for approved drugs 13); greater than 9 gave 93 (for approved drugs 12). Only two drugs in the database are both approved and have a predicted log P of greater than 10. These trends are a reflection of the fact that drugs having a high log P are difficult to get into the system (e.g., oral bioavailability).

The inherent difficulties in formulating hydrophobic drugs for oral administration is further exacerbated when large doses of drugs are required. The development of formulations for large doses of hydrophobic drugs for oral administration is particularly challenging as dosing regimens requiring administration of many unit dosage forms per day are undesirable and can lead to lack of patient compliance or adherence and other problems. Higher daily doses of hydrophobic drugs often require larger unit dosage forms, high pill burdens, or both. A number of studies have found that high pill burden reduces patient compliance and can lead to less than optimum treatment. Thus, there is a practical limit on (1) the size of a pill an individual can and will swallow and (2) the number of pills that can be taken daily. Solutions to these problems can require substantial innovation, especially in the context of hydrophobic drugs.

A challenge for developing formulations for hydrophobic drugs is related to drug loading. If a drug cannot be formulated with enough drug loading per unit dosage form, the pill burden or dosing regimen needed to achieve therapeutic efficacy can be impractical to the point of not being a feasible therapy or result in sub-optimal therapy. For example, there are reports in the literature that pill burden and patient compliance have an inverse relationship (see e.g., Wang et al. Nephrol Dial Transplant (2013) 0:1-9; Hardinger et al. Pharmacotherapy. 2012 May; 32(5):427-32; and Sax et al. PLoS One. 2012; 7(2):e31591). High drug loading of formulations for hydrophobic drugs can be problematic for a number of reasons. For example, high concentrations of the drug in the formulation can cause crystallization of the active agent (e.g., in the unit dosage form) and result in lower bioavailability.

One particular class of hydrophobic drugs exemplifies the difficulties in creating oral formulations of drugs that provide sufficient bioavailability, namely, testosterone esters. Numerous testosterone esters (testosterone cypionate, testosterone enanthate, testosterone undecanoate) are approved for treating testosterone deficiency but these are delivered by injection despite oral administration being highly preferable. In the United States, no orally administered testosterone ester is currently approved by the Food & Drug Administration.

In relation to steroid esters, one oral product that was withdrawn from the market consisted of testosterone undecanoate in oleic acid. Several problems were associated with this product including solubility problems, which required dosing regimens involving multiple capsules, each unit dosage form only having 40 mg of the active agent, and the low stability of this active agent in oleic acid. This product was withdrawn from the market and replaced by a new formulation in castor oil and lauroglycol. See Muchow et al. (2013) Journal of Pharmaceutical Technology & Drug Research (doi: 10.7243/2050-120X-2-4). Others have reported high drug loading issues with testosterone esters. For example, Dudley et al. revealed that drug loads higher than 23% of testosterone palmitate had undesirable properties (see WO2006113505).

Unlike any other ester of (8R,9S,10R,13S,14S,17S)-17-hydroxy-10,13-dimethyl-1,2,6,7,8,9,11,12,14,15,16,17-dodecahydrocyclopenta[a]phenanthren-3-one, (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate has unique physical-chemical characteristics (high lipophilicity and high melting point leading to low aqueous solubility) and unique pharmacokinetics/bioavailability upon oral dosing (e.g., half life, relative bioavailability to (8R,9S,10R,13S,14S,17S)-10,13-dimethyl-3-oxo-1,2,6,7,8,9,11,12,14,15,16,17-dodecahydrocyclopenta[a]phenanthren-17-yl undecanoate, Tₘₐₓ, etc.). Therefore, only a specific daily therapeutic dose in the range of 300-1500 mg is expected to be of therapeutic value in treating patients in need of (8R,9S,10R,13S,14S,17S)-17-hydroxy-10,13-dimethyl-1,2,6,7,8,9,11,12,14,15,16,17-dodecahydrocyclopenta[a]phenanthren-3-one therapy. These characteristics present a significant challenge in formulating compositions and dosage forms that allow for a practical number of dosage form units, such as capsules or tablets, per day that at the same time provides adequate bioavailability. Higher numbers of unit dosage forms/dose present significant patient compliance issues potentially compromising therapeutic efficacy. Inadequate solubility in the vehicle or poor release/bioavailability of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate in vivo could result in significant pill burden for patients in need of therapy. Typically, solubilized liquid formulations are desirable for adequate timely release of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate for desirable absorption. However, given the very limited solubility of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate in most common solvents (co-solvents and lipidic additives) of interest (See Table in Example 1), a liquid formulation for oral delivery has remained elusive.

There remains a significant unmet need to formulate (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate with lower pill burden per dose or on a daily basis, while maintaining adequate therapeutic blood levels when administered to subjects. We have surprisingly found that only certain specific compositions and dosage forms enable sufficient loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate that meets the lower daily pill burden requirement and have adequate release/bioavailability that is incapable of being estimated or computed based on available prior art.

### SUMMARY OF THE INVENTION

As is described herein, we have found that inclusion of specific carriers, additives or both, in pharmaceutical compositions or unit dosage forms provides adequate loading of (8R,9S,10R,135,14S,17S)-10,13-dimethyl-3-oxo-1,2,6,7,8,9,11,12,14,15,16,17-dodecahydrocyclopenta[a]phenanthren-17-yl tridecanoate. These pharmaceutical compositions and unit dosage forms translate to a lower pill burden and improved, patient friendly, dosing regimens.

In one configuration, the composition and dosage form allow for advantageous loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate (e.g., >23% w/w). The composition and dosage form can be formulated as a non-solid without any significant risk of precipitation upon storage (e.g., dissolution or release profile stable). The composition and dosage form can be formulated for suitable release that the inventors have found to be particularly desirable (e.g., at least 50% in two hours). The composition and dosage form provides adequate bioavailability (e.g., of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate, (8R,9S,10R,13S,14S,17S)-17-hydroxy-10,13-dimethyl-1,2,6,7,8,9,11,12,14,15,16,17-dodecahydrocyclopenta[a]phenanthren-3-one or both) while minimizing “pill burden” and avoiding complicated treatment regimens.

In one implementation, we have found that inclusion of additive (e.g., long chain fatty acids (e.g., saturated) or fatty acid glycerides) in the pharmaceutical composition or dosage form enables advantageous loading of active pharmaceutical ingredient (“API”) (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate with suitable release (e.g., greater than 50% in two hours). The inclusion of a long chain fatty acid (e.g., saturated) or fatty acid glyceride can allow for loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate of greater than 23% and no greater than 32% in the compositions and dosage forms. The composition and dosage form can be formulated as a non-solid and provides one or more of the following: reduced, little or no risk of precipitation upon storage (e.g., dissolution or release profile stable); lower pill burden; patient friendly dosing regimen; appropriate release or dissolution properties and suitable bioavailability. In this context, non-solid refers to a pharmaceutical composition that is not formulated or cannot be formulated as a tablet. At the same time, the pharmaceutical composition is not a liquid in the sense that the viscosity of the composition is increased over that of the carrier alone or is a liquid at temperatures elevated above ambient (e.g., above 20-23° C.). In a specific implementation, the pharmaceutical composition is flowable at temperatures that allow for the filling of soft gel capsules. At certain temperatures e.g., below ambient or below 15 or 10° C. the composition is more solid-like and is not flowable (e.g., cannot be used for filling soft gel capsules at this temperature). These compositions can be used for hard gel or soft gel capsules. These compositions comprise a liquid pharmaceutically acceptable carrier and an additive that allows for loading of the API above the solubility of the limit of the liquid carrier.

In another configuration, we have found that inclusion of a stabilizing agent in the composition and dosage form enables advantageous loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate. The stabilizing agent allows for valuable loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate (e.g., >23% w/w). The stabilizing agent allows the composition and dosage form to be formulated as a non-liquid and provides one or more of the following: reduced, little or no risk of precipitation upon storage (e.g., dissolution or release profile stable); lower pill burden; patient friendly dosing regimen; and suitable bioavailability. In a specific implementation, the pharmaceutical is flowable at temperatures that allow for the filling of hard gel capsules. At certain temperatures e.g., at ambient or below 15 or 10° C. the composition is more solid like and is not flowable (e.g., cannot be used for filling soft gel capsules). In one aspect, these compositions can be used for hard gel capsules. These compositions comprise a liquid pharmaceutically acceptable carrier and a stabilizing agent that allows for loading of the API above the solubility of the limit of the liquid carrier.

In yet another implementation, we have found that inclusion of stabilizing agent in the composition and dosage form enables advantageous loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate with adequate release properties (e.g., greater than 50% in 2 hours). The stabilizing agent allows the composition and dosage form to be formulated as a non-liquid and provides one or more of the following: lower daily pill burden; valuable loading e.g., greater than 23% and no greater than 32% w/w); reduced, little or no risk of precipitation upon storage (e.g., dissolution or release profile stable); adequate bioavailability; and patient friendly dosing regimen.

Described herein, the inventors have discovered compositions and dosages forms that, in some embodiments, allow for unexpectedly high drug loading for highly lipophilic drugs while maintaining excellent oral bioavailability. The pharmaceutical compositions and unit dosage forms described herein can reduce pill burden for hydrophobic drugs like (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. It was unexpectedly found that (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate can be formulated at advantageous drug loads (e.g., greater than 23%) while providing suitable bioavailability (e.g., capable of treating a hypogonadal male with less than 10 unit dosage forms per day) that allows for reduction in pill burden and accordingly improved patient adherence or compliance. Additionally, the composition (e.g., dosage form) has a release profile that is suitable for providing bioavailable API and the release profile is stable over time (e.g., under storage conditions).

According to one implementation, the pharmaceutical composition is a liquid (e.g., flowable) or the unit dosage form comprises a liquid pharmaceutical composition. For example, the unit dosage form can be a hard gel or soft gel capsule which contains a liquid pharmaceutical carrier in which the drug is dissolved (partially or fully). In a specific context, flowable refers to the ability of the composition to flow at a temperature of 40° C. With respect to liquid compositions, in one context, the liquid pharmaceutical carrier (like a C18 fatty acid having one unsaturation) is a liquid at ambient temperature (e.g., about 20 to about 25° C.). Typically, the liquid pharmaceutical compositions also include one or more additives that allow for increased loading of active pharmaceutical ingredient in the carrier beyond its solubility without substantial compromising dissolution (e.g., release), bioavailability or both. The one or more additives in specific aspects can increase the viscosity of the pharmaceutical composition. In specific configurations, the liquid pharmaceutical composition (e.g., carrier+additive+API (active pharmaceutical ingredient)) is a clear liquid at elevated temperatures (e.g., above 40° C., like 50° C.) flowable in the range of about 30° C. to about 40° C. and may be more hazy in appearance at ambient temperature having a paste or gel like consistency. In another implementation, the pharmaceutical composition is a liquid or the unit dosage form comprises a liquid. For example, the unit dosage form can be a hard gel or soft gel capsule which contains a liquid pharmaceutical carrier in which the drug is dissolved along with one or more additives. In a specific implementation, the unit dosage form is a soft gel capsule. According to a soft gel capsule implementation, the formulation (e.g., mixture of one or more pharmaceutical acceptable carriers and active pharmaceutical ingredient) is flowable at a temperature of less than 40° C. Exemplary carriers include fatty acids (e.g., C16-C18 having zero, one, two, or three unsaturations) and mono-, di-, and triglycerides thereof). According to this implementation, the composition or dosage form comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate.

According to another implementation, the pharmaceutical composition is a non-liquid (e.g., flowable) or the unit dosage form comprises a non-liquid pharmaceutical composition. For example, the unit dosage form can be a hard gel capsule which contains a liquid pharmaceutical carrier in which the drug is dissolved (partially or fully). In a specific context, flowable refers to the ability of the composition to flow at a temperature of 40° C. With respect to non-liquid compositions, in one context, pharmaceutical carrier (e.g., fatty acid, fatty acid glyceride, or combination thereof) is a liquid or solid at ambient temperature (e.g., about 20 to about 25° C.). Typically, the non-liquid pharmaceutical compositions also include one or more additives that allow for increased loading of active pharmaceutical ingredient in the carrier beyond its solubility without substantial compromising dissolution (e.g., release), bioavailability or both. The one or more additives in specific aspects can increase the viscosity of the pharmaceutical composition. In specific configurations, the non-liquid pharmaceutical composition (e.g., carrier+additive+API (active pharmaceutical ingredient)) is a clear liquid at elevated temperatures (e.g., above 40° C., like 50° C.) flowable in the range of about 30° C. to about 40° C. and may be more hazy in appearance at ambient temperature having a paste or gel like consistency. In another implementation, the pharmaceutical composition is a non-liquid or the unit dosage form comprises a non-liquid. For example, the unit dosage form can be a hard gel capsule which contains a non-liquid pharmaceutical carrier in which the drug is dissolved along with one or more additives. In a specific implementation, the unit dosage form is a hard gel capsule. According to a hard gel capsule implementation, the formulation (e.g., mixture of one or more pharmaceutically acceptable carriers and active pharmaceutical ingredient) is flowable at a temperature of greater than 35 or 40° C. and is not flowable at temperatures below 25° C. (e.g., not suitable for capsule filling at this temperature). Exemplary carriers include fatty acids (e.g., C16-C18 having zero, one, two, or three unsaturations) and mono-, di-, and triglycerides thereof. According to this implementation, the composition or dosage form comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate.

A specific example composition comprises a liquid or liquid-like carrier and an additive as well as API. The carrier is a liquid at ambient temperature in which API has sufficient solubility and the additive increases the amount of API that can be loaded into formulation without substantially compromising bioavailability or release. In a particular example, the composition comprises (a) octadecanoic acid, (9Z)-octadec-9-enoic acid, hexadecanoic acid, or a combination thereof (b) a mono-, di-, tri-propane-1,2,3-triol ester thereof or a combination thereof or (c) a combination thereof where the API is loaded in the range of 26% to 35% or 26% to 32%. In another particular example, the compositions comprises (a) octadecanoic acid, (9Z)-octadec-9-enoic acid, hexadecanoic acid or a combination thereof (b) a mono-, di-, tri-propane-1,2,3-triol ester thereof or a combination thereof, (c) 2-Isopropyl-5-methylcyclohexanone or (d) a combination thereof where the API is loaded in the range of 26% to 35% or 26% to 32%.

Another specific example composition comprises a carrier that is a liquid at ambient temperature (e.g., 20-23° C.) in which the API has sufficient solubility and an additive that prevents crystallization or recrystallization of API. In a particular example, the carrier comprises (9Z)-octadec-9-enoic acid and a compound of formula H—(O—CH₂—CH₂)ₙ—OH characterized as having an average molecular weight of about 600 to about 15,000 gram/mol where n is defined as giving said average molecular weight where the API is loaded in the range of 26% to 35% or 26% to 32%.

These specific compositions allow for delivery of 300 to 1500 mgs of API in 2-6 unit dosage forms (hard gel or soft gel capsules). Lower number of unit dosage form are provided with soft gel capsules (that provide adequate bioavailability for treating testosterone deficiency) which is a reflection of additional innovation because of the requirement of flowability of the composition at temperatures that allow filling (e.g., at 38° C. or less). These compositions provide excellent bioavailability for testosterone—see Examples included herein. The unit dosage forms are dissolution (release) profile stable over time (single point or profile), and release greater than 70% drug by 4 hours and greater than 50% at 2 hours.

### BRIEF DESCRIPTION OF THE FIGURES

FIG. 1 illustrates the release profile stability of exemplary compositions. See Example 9.

FIG. 2 illustrates the release profile stability of exemplary compositions. See Example 9.

FIG. 3 Illustrates a plot of the pharmacokinetic data derived from Example 11.

### DETAILED DESCRIPTION OF THE INVENTION

Formulations having advantageous loading of lipophilic active pharmaceutical ingredients (c log P of greater than 10) (e.g., (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate) are provided herein. The formulations having advantageous loading of drug described herein include pharmaceutical compositions, unit dosage forms having the pharmaceutical composition and intermediate compositions in the production of the high drug load formulations, pharmaceutical compositions and unit dosage forms described herein. The pharmaceutical compositions and unit dosage forms described herein are not solid. “Not solid” refers to a pharmaceutical composition that is flowable, semi-solid, a liquid, past, gel or liquid-like either at ambient temperature or processing temperatures that are suitable for hard gel of soft gel filling yet does not result in substantial degradation or decomposition of the active agent. “Solid” in this context refers to a composition that is a tablet (or caplet) or that can be formed into a tablet with acceptable characteristics (e.g., friability, hardness and disintegration).

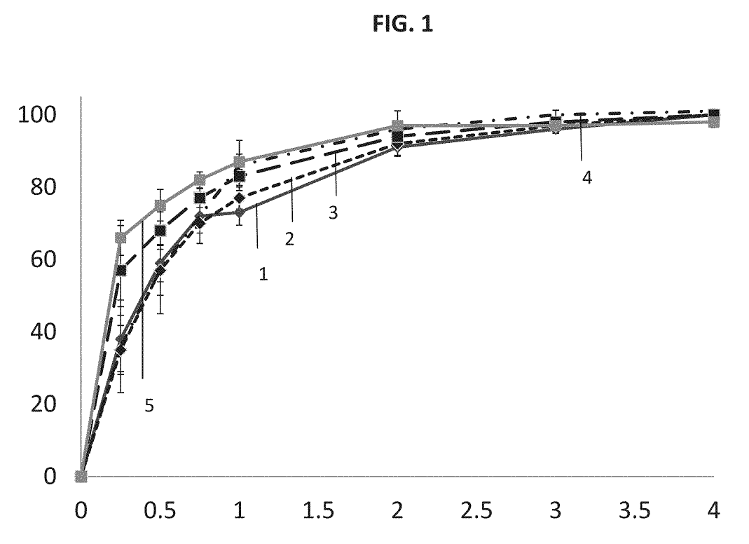

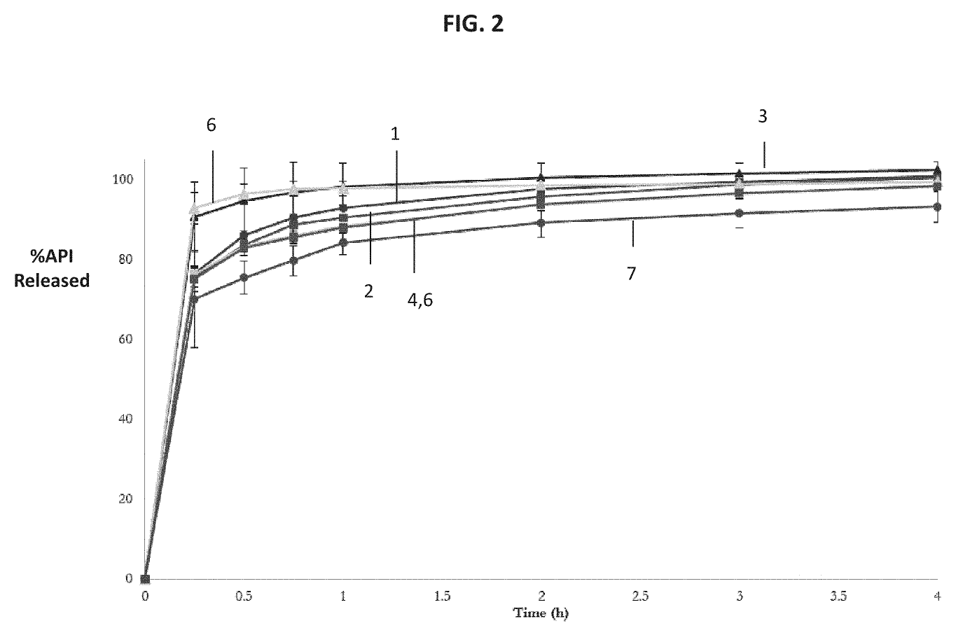

As described herein, the inventors have surprisingly found that advantageous drug load formulations of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate can be made that have excellent dissolution properties including release of substantially all of the active agent within 4 hours and are dissolution profile stable over a period of time. For example, FIG. 1 and FIG. 2 show formulations that release substantially all of the active agent at four hours (e.g., greater than 90% or 95%). Formulation types that do not release substantially all of the active agent at four hours are described in Example 13.

It was unexpectedly found that liquid carriers can have improved drug loading (e.g., greater than the solubility of the drug in the liquid carrier) when formulated in a liquid carrier having one or more additives without substantially compromising dissolution or bioavailability. In one specific embodiment, the formulations or compositions (e.g., active agent+liquid carrier+one or more additives) are flowable at temperatures (e.g., 40° C. or less) that allow for production (e.g., filling) of soft gel capsule unit dosage forms without substantially comprising the structural integrity of the capsule or API. It appears that the combination of carriers, additives, and API provide a synergistic or unexpected effect that allows for loading above the solubility of the API in the carrier and provides suitable bioavailability.

It should be noted that, the singular forms “a,” “an,” and, “the” include plural references unless the context clearly dictates otherwise. Thus, for example, reference to “an excipient” includes reference to one or more of such excipients, and reference to “the carrier” includes reference to one or more of such carriers.

As used herein, “active pharmaceutical ingredient” or “API” refers to (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. In the context of this disclosure, the API is the substance in the pharmaceutical composition or unit dosage forms described herein. The biological active metabolite of both these API's is (8R,9S,10R,13S,14S,17S)-17-hydroxy-10,13-dimethyl-1,2,6,7,8,9,11,12,14,15,16,17-dodecahydrocyclopenta[a]phenanthren-3-one (e.g., testosterone which is produced in vivo by de-esterification. Another important biological active metabolite is (17-β)-hydroxy-5α-androstan-3-one with an IUPAC name of (5S,8R,9S,10S,13S,14S,17S)-17-hydroxy-10,13-dimethyl-1,2,4,5,6,7,8,9,11,12,14,15,16,17-tetradecahydrocyclopenta[a]phenanthren-3-one (CAS No. 521-18-6).

(8R,9S,10R,13S,14S,17S)-17-Hydroxy-10,13-dimethyl-1,2,6,7,8,9,11,12,14,15,16,17-dodecahydrocyclopenta[a]phenanthren-3-one is also known as testosterone and has a CAS number of 58-22-0. Thus, the active pharmaceutical ingredient (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate is testosterone esterified with tridecanoic acid (IUPAC name for the alkanoic acid having CAS number 638-53-9).

As used herein, the term “Triton X100” or Triton “X-100” is a non-ionic detergent and refers to a composition as known as polyethylene glycol p-(1,1,3,3-tetramethylbutyl)-phenyl ether, octyl phenol ethoxylate, polyoxyethylene octyl phenyl ether, 4-octylphenol polyethoxylate, Mono 30, TX-100, t-octylphenoxypolyethoxyethanol, or Octoxynol-9 and associated with CAS NO. 9002-93-1.

A “pharmaceutical composition” as used herein refers to a composition comprising or prepared from API and a pharmaceutically acceptable carrier, excipient, additive, stabilizing agent or combination thereof. A “unit dosage form” as used herein refers to a medicament prepared from or comprising a pharmaceutical composition and includes tablets, capsules, caplets, gel caps, ampoules, suspensions, solutions, gels, dispersions and other dosage units typically associated with parenteral, enteral, topical or other forms of administration of an API to a subject in need thereof.

As used herein, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in the specification, like “comprising” or “including,” it is understood that direct support should be afforded also to “consisting essentially of” language as well as “consisting of” language as if stated explicitly and vice versa.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The terms “serum testosterone” or “serum (17-β)-Hydroxy-4-Androsten-3-one levels,” “serum T levels,” “serum testosterone concentration,” “plasma testosterone concentration,” “testosterone concentration in the blood,” and “serum testosterone concentration,” are used interchangeably and refer to the “total” testosterone concentration which is the sum of the bioavailable testosterone including free and bound testosterone concentrations. Unless otherwise specified, these values are “observed” testosterone concentrations without adjusting or correcting for the base-line serum testosterone levels in the subject(s). As with any bio-analytical measure, for increased consistency, the method employed to measure initial serum testosterone levels should be consistent with the method used to monitor and re-measure serum testosterone levels during clinical testing and testosterone therapy for a subject. Unless otherwise stated, “testosterone concentration” refers to serum total testosterone concentration.

Average serum testosterone concentrations can be determined using methods and practices known in the art. For example, the average baseline plasma testosterone concentration of a human male is the arithmetic mean of the total plasma testosterone concentration determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As used herein, the term “AUCₜ₁₋ₜ₂” is the area under the curve of a plasma-versus-time graph determined for the analyte from the time “t1 to time t2”. Wherein t1 and t2 are times (in hours) post dosing. For Example, t1 could be 1 hour and t2 could be 2 hours. As used herein, the term “C_{avg},” “Cₐᵥₑ,” or “C-average” are used interchangeably, and is determined as the AUCₜ₁₋ₜ₂ mean AUC divided by the time period (|t1-t2|). For example, C_{avg t0-t8} is the average plasma concentration over a period of 8 hours from t1=0 to t2=8 hours) post-dosing determined by dividing the AUC t₀₋ₜ₈ value by 8. Similarly, C_{avg t0-t12} is the average plasma concentration over a period of 12 hours post-dosing determined by dividing the AUCt₀₋ₜ₁₂ value by 12 (t1=0-t2=12). Similarly, C_{avg t12-t24} is the average plasma concentration over a period of 12 hours post-dosing determined by dividing the AUCtₜ₂₋ₜ₂₄ value by 12 (t1=12-t2=24), C_{avg-t24} is the average plasma concentration over a period of 24 hours post-dosing determined by dividing the AUCₜ₀₋ₜ₂₄ value by 24 (t1=0-t2=24), and so on. Unless otherwise stated, all C_{avg} values are considered to be C_{avg-t24} and unless otherwise stated, all the time values are expressed in hours (h). For example, the term C_{avg t0-t24} denotes C_{avg} from time zero (0) to 24 hours post dosing.

In one embodiment, a pharmaceutical composition is provided. According to this embodiment, the pharmaceutical composition comprises an active agent which is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate, a liquid carrier, and one or more additives. The solubility of the active agent in the carrier (without any additive or stabilizing agent) is greater than 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, or 150 mg/mL at a selected temperature. According to this embodiment, the selected temperature is chosen from 10° C., 15° C., 20° C., 25° C., 30° C., 35° C., 40° C., 45° C., 50° C., 55° C., 60° C., 65° C., 70° C., 75° C., 80° C., or 85° C. According to this embodiment, the selected temperature is chosen from about 32° C., 33° C., 34° C., 35° C., 36° C., 37° C., 38° C., 39° C., or 40° C. In a specific aspect of this embodiment, the liquid carrier is a pharmaceutically acceptable carrier. The pharmaceutical composition of this embodiment further comprises one or more additives. The one or more additives improve the drug loading of the pharmaceutical composition without substantially compromising bioavailability, the dissolution profile, dissolution profile stability, or a combination thereof. By way of example, consider a liquid carrier (without any additives) where the solubility of the active agent is 40 mg/mL at a selected temperature (e.g., 20° C.). Unit dosage form (A) has 40 mg of active agent in 1 mL of this carrier (without any additives). A similar unit dosage form (B) has 80 mg of active agent (40 mg solubilized and 40 mg not solubilized (e.g., crystalline)) in 1 mL of this carrier. Another unit dosage form (C) has 80 mg of active agent in 1 mL of the carrier and additionally one or more additives as described herein. Two unit dosage forms of (A) have the equivalent amount of active agent as one unit dosage form of (B) or (C). According to this embodiment, the bioavailability, dissolution (release) profile, dissolution (release) profile stability or a combination thereof, of unit dosage form (B) is substantially compromised compared to that of two (A) unit dosage forms or a (C) unit dosage form.

As used herein, the term “substantially compromised” in some contexts refers to a difference of more than 5%, 10%, 15%, 20% or 25% when tested under identical or similar conditions. For example, consider a single time point dissolution value e.g, 1 hour. “Substantially compromised” would be when the dissolution of the test composition is e.g., 15% less than the reference composition at one hour when tested under identical or similar conditions. In reference to a dissolution profile, the value at 2, 3, 4, or 5 or more time points must differ by more than 5%, 10%, 15%, 20% or 25% when tested under identical or similar conditions. In reference to bioavailability, a difference of more than 5%, 10%, 15%, 20% or 25% when tested under identical or similar conditions (same dosing of active agent) is substantially compromised.

In one embodiment, a pharmaceutical composition is provided that has advantageous loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate which is in a physical form that has a higher energy state than crystalline (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate, respectively. In this context, higher energy state than crystalline refers to e.g., partially or fully solubilized (higher energy) vs. crystalline (lower energy) or amorphous (higher energy) vs. crystalline (lower energy). According to one aspect of this embodiment, provided herein is a composition having one or more pharmaceutically acceptable carriers, one or more pharmaceutically acceptable additives, or both, and amounts of these components that allow for advantageous loading of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate in the pharmaceutical composition. The solubility of the drug in the carrier is typically greater than 5 mg/mL, 50 mg/mL or 100 mg/mL and is typically less than 300 mg/mL. The additive allows for an increase in the amount of drug loading without substantially comprising release, bioavailability or both. The one or more carriers, one or more additives, or both, and concentrations (or amounts) of these agents, and concentrations (or amounts) of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate provide unexpectedly good bioavailability in a minimal number of unit dosage forms. The formulations also are dissolution profile stable. These high drug loading formulations allow for greater than 23%, 24%, 25%, 26%, 27%, 28%, or 29% w/w drug loading. For example, daily doses sufficient to provide substantial therapeutic effects in hypogonadal males e.g., serum (8R,9S,10R,13S,14S,17S)-17-Hydroxy-10,13-dimethyl-1,2,6,7,8,9,11,12,14,15,16,17-dodecahydrocyclopenta[a]phenanthren-3-one levels (e.g., C_{avg}) of greater than 300 ng/dL, can be achieved by the administration of 1, 2, 3, 4, 5, or 6 unit dosage forms as described herein. The unit dosage forms described herein can have 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg or more of 8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Alternatively, the unit dosage form can have from 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 525 mg to 550 mg, 550 mg to 575 mg, 575 mg to 600 mg of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate in a form that provides excellent of bioavailability. In one aspect, the pharmaceutical composition is encapsulated (hard gel or soft gel).

In addition to providing the (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate in a form that has excellent bioavailability, the composition or dosage form allows for greater than 50%, 70% or 85% release in 1, 2, 3 or 4 hours at most. Moreover, the compositions and dosage forms are dissolution or release profile stable. As used herein, “release profile stable” or “dissolution profile stable” refers to the ability of a unit dosage form to retain a substantially similar release profile (at two or more time points separated by at least 15 minutes e.g., 2, 3, 4, 5 time points) or a single time point (e.g., 15, 30, 60, 90, 120, 180 or 240 minutes) percent (less than 5%, 10%, 15% or 20% change) release over a period of time (e.g., 1 day, 1 week, 1 month, 2 months, 3 months, 6 months, 1 year, or two years or more) when stored under particular temperature conditions in the range of 15° C. to 65° C. (e.g., 10° C., 15° C., 20° C., 25° C., 30° C., 35° C., 40° C., 45° C., 60° C., or 65° C.).

In one embodiment, the pharmaceutical composition has, on a weight to weight basis, from about 10% to about 50% of 8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate and about 10% to about 90% of (a) octadecanoic acid, (9Z)-octadec-9-enoic acid, hexadecanoic acid or a combination thereof and (b) a mono-, di-, tri-propane-1,2,3-triol ester thereof, a combination of mono-, di-, or tri-propane-1,2,3-triol esters thereof or (c) a combination thereof. The pharmaceutical composition can be encapsulated e.g., soft gel or hard gel. The pharmaceutical composition may include a stabilizing agent, optional additive or both.

Typically the fatty acids discussed herein and their esters (e.g., of glycerides (a mono-, di-, tri-propane-1,2,3-triol ester)) are “C8 to C22 fatty acid(s)” and as referred to herein refers to both saturated fatty acids (e.g., having no carbon-carbon double bonds) and unsaturated fatty acids (e.g., having one or multiple carbon-carbon double bonds (e.g., 2, 3, 4 or 5 or more) wherein the molecule or moiety has from 8 to 22 carbons. Sub-ranges are also specified herein, for example, C16-C18 fatty acid refers to 16, 17, 18 carbon fatty acids. Examples of saturated C8 to C22 fatty acids include, but are not limited to and can be chosen from octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, and tetracosanoic acid. Examples of unsaturated C8 to C22 fatty acids can be chosen from, 9Z-octadecenoic acid, 5Z,8Z,11Z,14Z-eicosatetraenoic acid, 2Z-octenoic acid, 2E-octenoic acid, 3Z-octenoic acid, 3E-octenoic acid, 4Z-octenoic acid, 5Z-octenoic acid, 5E-octenoic acid, 6Z-octenoic acid, 6E-octenoic acid, 2Z-nonenoic acid, 3-nonenoic acid, 8-nonenoic acid, 2-decenoic acid, 3-decenoic acid, 4E-decenoic acid, 8-decenoic acid, 9-decenoic acid, 2-undecenoic acid, 9-undecenoic acid, 10-undecenoic acid, 2-dodecenoic acid, 4-dodecenoic acid, 6-dodecenoic acid, 7-dodecenoic acid, 9-dodecenoic acid, 10-dodecenoic acid, 11-dodecenoic acid, 2-tridecenoic acid, 11-tridecenoic acid, 12-tridecenoic acid, 2-tetradecenoic acid, 4-tetradecenoic acid, 8Z-tetradecenoic acid, 9Z-tetradecenoic acid, 2-pentadecenoic acid, 14-pentadecenoic acid, 2-hexadecenoic acid, 7-hexadecenoic acid, 9Z-hexadecenoic acid, 9E-hexadecenoic acid, 10Z-hexadecenoic acid, 2-heptadecenoic acid, 9Z-heptadecenoic acid, 2Z-octadecenoic acid, 2Z-octadecenoic acid, 3-octadecenoic acid, 4-octadecenoic acid, 5E-octadecenoic acid, 6Z-octadecenoic acid, 6E-octadecenoic acid, 7Z-octadecenoic acid, 7E-octadecenoic acid, 8Z-octadecenoic acid, 8E-octadecenoic acid, 9E-octadecenoic acid, 10Z-octadecenoic acid, 10E-octadecenoic acid, 11Z-octadecenoic acid, 11E-octadecenoic acid, 12Z-octadecenoic acid, 12E-octadecenoic acid, 15E-octadecenoic acid, 16E-octadecenoic acid, 17Z-octadecenoic acid, 2-nonadecenoic acid, 9Z-eicosenoic acid, 11Z-eicosenoic acid, 11E-eicosenoic acid, 14Z-eicosenoic acid, 11Z-docosenoic acid, 13Z-docosenoic acid, 13E-docosenoic acid, 22-tricosenoic acid, 15Z-tetracosenoic acid, 15E-tetracosenoic acid, 2E,4Z-decadienoic acid, 2E,4E-decadienoic acid, 2Z,6Z-decadienoic acid, 2E,6Z-decadienoic acid, 2E,6E-decadienoic acid, 4E,6E-decadienoic acid, 9,12-hexadecadienoic acid, 5Z,12Z-otadecadienoic acid, 5Z,12E-otadecadienoic acid, 5E,12Z-octadecadienoic acid, 5E,12E-octadecadienoic acid, 6,8-octadecadienoic acid, 8E,10E-octadecadienoic acid, 8Z,11Z-octadecadienoic acid, 9Z,11Z-octadecadienoic acid, 9Z,11E-octadecadienoic acid, 9E,11E-octadecadienoic acid, 9Z,12Z-octadecadienoic acid, 9Z,12E-octadecadienoic acid, 9E,12Z-octadecadienoic acid, 9E,12E-octadecadienoic acid, 10Z,12Z-octadecadienoic acid, 10E,12Z-octadecadienoic acid, 10E,12E-octadecadienoic acid, 10Z,13Z-octadecadienoic acid, 10Z,13Z-nonadecadienoic acid, 11,14-eicosadienoic acid, 5,13-docosadienoic acid, 13,16-docosadienoic acid, 17,20-hexacosadienoic acid, 4,7,10-hexadecatrienoic acid, 5E,8E,11E-hexadecatrienoic acid, 6,9,12-hexadecatrienoic acid, 6,10,14-hexadecatrienoic acid, 7Z,10Z,13Z-hexadecatrienoic acid, 9,12,15-hexadecatrienoic acid, 3E,9Z,12Z-octadecatrienoic acid, 6Z,9Z,12Z-octadecatrienoic acid, 6,10,14-octadecatrienoic acid, 8Z,10E,12Z-octadecatrienoic acid, 8E,10E,12Z-octadecatrienoic acid, 8E,10E,12E-octadecatrienoic acid, 9Z,11E,13Z-octadecatrienoic acid, 9Z,11E,13E-octadecatrienoic acid, 9E,11E,13E-octadecatrienoic acid, 9,12,14-octadecatrienoic acid, 9Z,12Z,15Z-octadecatrienoic acid, 9E,12E,15E-octadecatrienoic acid, 10,12,14-octadecatrienoic acid, 10E,12E,14E-octadecatrienoic acid, 10,12,15-octadecatrienoic acid, 5,8,11-eicosatrienoic acid, 8Z,11Z,14Z-eicosatrienoic acid, 11,14,17-eicosatrienoic acid, 7,10,13-docosatrienoic acid, 8,11,14-docosatrienoic acid, 4Z,7Z,10Z,13Z-hexadecatetraenoic acid, 4,7,11,14-hexadecatetraenoic acid, 4,8,12,16-hexadecatetraenoic acid, 6,9,12,15-hexadecatetraenoic acid, 3E,9Z,12Z,15Z-octadecatetraenoic acid, 6,9,12,15-octadecatetraenoic acid, 9E,11E,13E,15E-octadecatetraenoic acid, 9,12,15,17-octadecatetraenoic acid, 4,8,12,16-eicosatetraenoic acid, 6,10,14,18-eicosatetraenoic acid, 8,11,14,17-eicosatetraenoic acid, 4,7,10,13-docosatetraenoic acid, 7Z,10Z,13Z,16Z-docosatetraenoic acid, 8,12,16,19-docosatetraenoic acid, 4,8,12,15,18-eicosapentaenoic acid, 4,7,10,13,16-docosapentaenoic acid, 4,8,12,15,19-docosapentaenoic acid, 7,10,13,16,19-docosapentaenoic acid, 4Z,7Z,10Z,13Z,16Z,19Z-docosahexaenoic acid, 4,8,12,15,19,21-tetracosahexaenoic acid, 5-(2-cyclopentenyl)-pentanoic acid, 7-(2-cyclopentenyl)-heptanoic acid, 9-(2-cyclopentenyl)-nonanoic acid, 3Z-decenoic acid, 4Z-decenoic acid, 2,3-decadienoic acid, 2,5-decadienoic acid, 2E,7E-decadienoic acid, 2E,7Z-decadienoic acid, 2Z,6E-decadienoic acid, 3,4-decadienoic acid, 3,5-decadienoic acid, 3E,5Z-decadienoic acid, 3Z,5E-decadienoic acid, 4,8-decadienoic acid, 4E,9-decadienoic acid, 5E,9-decadienoic acid, 5E,8E-decadienoic acid, 6E,8E-decadienoic acid, 7,9-decadienoic acid, 2E,6E,8E-decatrienoic acid, 2E,6Z,8E-decatrienoic acid, 2Z-undecenoic acid, 3E-undecenoic acid, 6Z-undecenoic acid, 8Z-undecenoic acid, 9Z-undecenoic acid, 2E,4E-undecadienoic acid, 10Z-dodecenoic acid, 2Z-dodecenoic acid, 5E-dodecenoic acid, 5Z-dodecenoic acid, 6Z-dodecenoic acid, 7Z-dodecenoic acid, 9Z-dodecenoic acid, 2E,4E-dodecadienoic acid, 2E,6Z-dodecadienoic acid, 2E,8E-dodecadienoic acid, 2E,8Z-dodecadienoic acid, 2Z,8E-dodecadienoic acid, 2Z,8Z-dodecadienoic acid, 5E,7E-dodecadienoic acid, 7Z,9E-dodecadienoic acid, 8E,10E-dodecadienoic acid, 8Z,10E-dodecadienoic acid, 2E,4E,8Z,10E-dodecatetraenoic acid, 2E,6E,8E,10E-dodecatetraenoic acid, 2E,6E,8Z,10E-dodecatetraenoic acid, 3,5,7,9,11-dodecapentaenoic acid, 3E,5E-tridecadienoic acid, 3Z,5E-tridecadienoic acid, 3E-tetradecenoic acid, 4Z-tetradecenoic acid, 5Z-tetradecenoic acid, 7Z-tetradecenoic acid, 9E-tetradecenoic acid, 10Z,12E-tetradecadienoic acid, 2E,4E-tetradecadienoic acid, 3,4-tetradecadienoic acid, 3Z,5E-tetradecadienoic acid, 3Z,5Z-tetradecadienoic acid, 5,8-tetradecadienoic acid, 5Z,8Z-tetradecadienoic acid, 5,7,9,11,13-tetradecapentaenoic acid, 10Z-pentadecenoic acid, 10-hexadecenoic acid, 11-hexadecenoic acid, 11Z-hexadecenoic acid, 13-hexadecenoic acid, 13Z-hexadecanoic acid, and 2Z-hexadecenoic acid. It is noted that the term C8 to C22 is intended to include both branched chain versions, cyclic versions, as well as straight versions as long as the total number of carbons of the fatty acid is in the range of from 8 carbons to 22 carbons. Preferred amongst the C8 to C22 fatty acids described in this paragraph are those that are pharmaceutically acceptable. Even more preferred amongst the C8 to C22 fatty acids described in this paragraph are those that are pharmaceutically acceptable to one or more regulatory agencies such as the United States Food and Drug Agency (e.g., inactive ingredients in approved drug products; or 1. determination by FDA that the substance is “generally recognized as safe” (GRAS) pursuant to Title 21, U.S. Code of Federal Regulations, Parts 182, 184 or 186 (21 CFR 182, 184 & 186), 2. approval of a food additive petition as set forth in 21 CFR 171; or 3. the excipient is referenced in, and part of, an approved new drug application (NDA) for a particular function in that specific drug product), the European Medicines Agency, the Japanese Pharmaceuticals and Medical Devices, or is listed in a Pharmacopoeia (e.g., European, United States, Japan, International, or one in the World Health Organization Index of Pharmacopoeias). In one aspect, preferred C8 to C22 fatty acids are chosen from C14 to C20 fatty acids. In one aspect, preferred C8 to C22 fatty acids are selected from octadecanoic acid, (9Z)-octadec-9-enoic acid, hexadecanoic, (9Z,12Z)-9,12-octadecadienoic acid, or hexadec-9-enoic acid. In one aspect, preferred C8 to C22 fatty acids are selected from octadecanoic acid, (9Z)-octadec-9-enoic acid, hexadecanoic or hexadec-9-enoic acid.

As used herein, “a mono-, di-, tri-propane-1,2,3-triol ester” refers to a fatty acid ester or fatty acid esters (e.g., two or more (combination of esters or mixtures of esters)) of propane-1,2,3-triol. It is noted that the same propane triol can have different ester moieties (derived from chemically distinct fatty acids). In this context fatty acid is as defined above e.g., C8 to C22 fatty acids. In a specific definition, C8 to C22 fatty acids that form the ester moiety or moieties of the mono-, di-, propane triol ester are selected from octadecanoic acid, (9Z)-octadec-9-enoic acid, hexadecanoic acid, (9Z,12Z)-9,12-octadecadienoic acid, or hexadec-9-enoic acid. In another specific definition, the mono-, di-, tri-propane-1,2,3-triol ester is 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1, 2, 3-trihexadecanoyl-glycerol, 1,2,3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol or 1,3-dihexadecanoyl-2-octadecanoyl-glycerol. In another specific aspect, at least 50%, 60%, 70%, 80% or 85% of the total content of mono-, di-, tri-propane-1,2,3-triol ester is 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1,2,3-trihexadecanoyl-glycerol, 1,2,3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol, 1,3-dihexadecanoyl-2-octadecanoyl-glycerol or a combination thereof. In another specific definition, the combination of mono-, di-, tri-propane-1,2,3-triol ester are esters of octadecanoic acid, hexadecanoic acid, or a combination thereof. In another specific definition the combination of mono-, di-, tri-propane-1,2,3-triol ester are esters of octadecanoic acid, hexadecanoic acid, or a combination thereof with a triglyceride content of from 30% to 60%, a diglyceride content of 25% to 55% and a monoglyceride content of 4% to 15%.

In specific embodiments and compositions described herein, free fatty acids are employed (e.g., as a carrier or solvent for API). When such compounds are referred to herein, it is understood that such compounds often are not 100% one particular free fatty acid. Thus, reference to a specific free fatty acid refers to a compound that is at least 50% of a specific free fatty acid by weight, preferably at least 55%, 60%, 65%, 70%, 75%, 80%, 85% or 90%. In one specific, the fatty acid is (9Z)-octadec-9-enoic acid which is at least 55%, 60%, 65%, 70%, 75%, 80%, 85% or 90% (9Z)-octadec-9-enoic acid. For example, at least 65% or at least 85% (9Z)-octadec-9-enoic acid. In one specific aspect, (9Z)-octadec-9-enoic acid is about 63%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In another specific aspect, (9Z)-octadec-9-enoic acid is is about 70%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about 80%-100% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about70%-95% (9Z)-octadec-9-enoic acid, less than 7% tetradecanoic acid, less than 18% hexadecanoic acid, less than 10% (9Z)-hexadec-9-enoic acid, less than 8% octadecanoic acid, less than 20% (9Z,12Z)-9,12-octadecadienoic acid, less than 6% linolenic acid and less than 5% fatty acid with chain length greater than 18 carbons. In one specific aspect, (9Z)-octadec-9-enoic acid is about 75%-95% (9Z)-octadec-9-enoic acid, less than 4% tetradecanoic acid, less than 14% hexadecanoic acid, less than 6% (9Z)-hexadec-9-enoic acid, less than 4% octadecanoic acid, less than 16% (9Z,12Z)-9,12-octadecadienoic acid, less than 4% linolenic acid and less than 3% fatty acid with chain length greater than 18 carbons. In a specific aspect, (9Z)-octadec-9-enoic acid has one or more of the following, more than 0.1% tetradecanoic acid, more than 0.1% hexadecanoic acid, more than 0.1% (9Z)-hexadec-9-enoic acid, more than 0.1% octadecanoic acid, more than 0.1% (9Z,12Z)-9,12-octadecadienoic acid, more than 0.1% linolenic acid and more than 0.1% fatty acid with chain length greater than 18 carbons. In a specific aspect, (9Z)-octadec-9-enoic acid is greater than 80 or 85% (9Z)-octadec-9-enoic acid has one or more of the following, 0.1-5% tetradecanoic acid, 0.1-16%% hexadecanoic acid, 0.1-8% (9Z)-hexadec-9-enoic acid, 0.1-6% octadecanoic acid, more than 0.118% (9Z,12Z)-9,12-octadecadienoic acid, 0.1-4% linolenic acid and 0.1-4% fatty acid with chain length greater than 18 carbons. In a specific aspect, (9Z)-octadec-9-enoic acid has a melting point in the range of about 4-14° C. or about 6-12° C. In a specific aspect, (9Z)-octadec-9-enoic acid is from a vegetable source. In a specific aspect, (9Z)-octadec-9-enoic acid is from an edible source.

It has been found that (9Z)-octadec-9-enoic acid preparation can effect the stability and performance of oral pharmaceutical composition comprosing this carrier. Thus, the formulation, compositions and dosage forms described herein that contain (9Z)-octadec-9-enoic acid preferably contain a preparation as described in the Table below and subsequent two paragraphs.

TABLE
Different percent content of components of (9Z)-octadec-9-enoic acid (ratio
of (9Z)-Octadec-9-enoic acid to other fatty acid in parentheses)

| | (9Z)- | | | (9Z)- | | | | |
|---|---|---|---|---|---|---|---|---|
| | Octadec- | | | Hexadec- | | (9Z,12Z)-9,12- | | C18+ |
| | 9-enoic | Tetradecanoic | Hexadecanoic | 9-enoic | Octadecanoic | Octadecadienoic | Linolenic | Fatty |
| | acid | acid | acid | acid | acid | acid | acid | Acid |
| | % | % | % | % | % | % | % | % |
| 1 | 50 | 5 | 2 | 5 | 20 | 10 | 5 | 3 |
| | | | | | (2.5:1) | | | |
| 2 | 55 | 4 | 10 | 6 | 15 | 5 | 3 | 2 |
| | | | | | (3.67:1) | | | |
| 3 | 60 | 3 | 23 | 2 | 2 | 5 | 3 | 2 |
| | | | (2.6:1) | | | | | |
| 4 | 65 | 3 | 7 | 5 | 10 | 4 | 3 | 3 |
| | | | | | (6.5:1) | | | |
| 5 | 75 | 1 | 2 | 3 | 15 | 2 | 1 | 1 |
| | | | | | (5:1) | | | |
| 6 | 65 | 3 | 8 | 3 | 10 | 4 | 3 | 4 |
| | | | | | (6.5:1) | | | |
| 7 | 65 | 3 | 10 | 4 | 3 | 7 | 2 | 1 |
| 8 | 75 | 2 | 3 | 4 | 1 | 9 | 3 | 3 |
| 9 | 80 | 3 | 3 | 1 | 6 | 2 | 4 | 1 |
| 10 | 85 | 2 | 2 | 3 | 4 | 2 | 1 | 1 |
| 11 | 86 | 2 | 2 | 2 | 2 | 2 | 2 | 2 |
| 12 | 88 | 1 | 1 | 1 | 6 | 1 | 1 | 1 |
| 13 | 90 | 1 | 1 | 2 | 3 | 1 | 1 | 1 |
| 14 | 93 | 1 | 1 | 1 | 1 | 1 | 1 | 1 |
| 15 | 95 | 0 | 1 | 1 | 1 | 1 | 1 | 0 |
| 16 | 70 | 0 | 0 | 0 | 0 | 23 | 7 | 0 |
| | | | | | | (3:1) | (10:1) | |
| 17 | 68 | 0 | 0 | 0 | 0 | 22 | 10 | 0 |
| | | | | | | (3.1:1) | (6.8:1) | |

In the Table above, numbers 7-15 are considered to have desirable properties whereas numbers 1-6 are considered to have undesirable properties. Undesirable properties include, but are not limited to, undesirable dissolution or release profiles (or stability thereof) of API from formulations containing (9Z)-octadec-9-enoic acid, undesirable stability of API in formulation containing (9Z)-octadec-9-enoic acid, undesirable solubility of API in formulations containing (9Z)-octadec-9-enoic acid, undesirable manufacturing properties for formulations containing (9Z)-octadec-9-enoic acid or a combination thereof.

In some embodiments, it was also found that particular ratios of components of (9Z)-octadec-9-enoic acid are desirable or undesirable (e.g., having desirable or undesirable properties such as those described in the paragraph above). For example, a ratio (9Z)-octadec-9-enoic acid:Octadecanoic acid of 10:1 or greater is desirable; a ratio (9Z)-octadec-9-enoic acid:hexadecanoic acid of 4:1 or greater is desirable; a ratio of unsaturated:unsaturated C14-C18 fatty acid of greater than 2:1 is desirable. In related examples, a ratio of (9Z)-octadec-9-enoic acid:octanoic acid of greater than 4:1 is desirable; a ratio of (9Z)-octadec-9-enoic acid:octanoic acid of less than 4:1 is undesirable; a ratio of (9Z)-octadec-9-enoic acid:hexanoic acid of greater than 4:1 is desirable; a ratio of (9Z)-octadec-9-enoic acid:decanoic acid of greater than 4:1 is desirable. It has also been found that higher ratios of (9Z)-Octadec-9-enoic acid to (9Z,12Z)-9,12-Octa-decadienoic acid, linolenic acid or both are desirable as increased levels of components lead to undesirable properties such as chemical instability of API, physical instability of the composition or unit dosage form, undesirable dissolution or release profiles (or stability thereof) of API from formulations or a combination thereof. For example, a ratio of (9Z)-octadec-9-enoic acid: (9Z,12Z)-9,12-Octa-decadienoic acid of less than 4:1, 3.8:1, or 3.5:1 is undesirable. For example, a ratio of (9Z)-octadec-9-enoic acid:linolenic acid of less than 20:1, 17:1, or 15:1 is undesirable. Thus, Examples 16 and 17 in the Table above are undesirable.

In one embodiment, the (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate active pharmaceutical ingredient as described in this paragraph is further part of a composition having one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—CH₂)ₙ—OH where n is an integer from 3 to 900. In a more specific aspect of this embodiment, the 8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate active pharmaceutical ingredients as described in this paragraph are further part of a composition having one or more components chosen from the following:
(A) octadecanoic acid, (9Z)-octadec-9-enoic acid, (9Z,12Z)-9,12-octadecadienoic acid, or hexadecanoic acid;
(B) a mono-, di-, or tri-propane-1,2,3-triol ester of (A);
(C) a combination of mono-, di-, or tri-propane-1,2,3-triol esters of (A);
(D) a combination of one or more of (A)-(C);
(E) (2S,5R)-2-Isopropyl-5-methylcyclohexanone, acetic acid [(1R,2S,5R)-2-isopropyl-5-methylcyclohexyl]ester; (1R,2S,5R)-2-isopropyl-5-methylcyclohexanol or a combination thereof;
(F) 2-isopropyl-5-methylcyclohexanol, 2-Isopropyl-5-methylcyclohexanone, acetic acid [(2-isopropyl-5-methylcyclohexyl] ester or a combination thereof.
(G) Polyoxyethylated oil;
(H) Polyoxyethylated hydrogenated vegetable oil;
(I) Polyoxyethylated hydrogenated vegetable oil;
(J) Polyoxyethylated hydrogenated castor oil;
(K) H—(O—CH₂—CH₂)ₙ—OH where n is an integer from 5 to 600;
(L) a branched; star, or comb analog (in this specific context analog refers to a molecule having the same molecular weight or average molecular weight) of H—(O—CH₂—CH₂)ₙ—OH where n is an integer from 5 to 600; and
(M) polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, hydroxypropylmethyl cellulose acetate succinate, or a combination thereof. The pharmaceutical composition has (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate at greater than 23%, 24%, 25%, 26%, 27%, 28%, or 29% w/w drug loading (and less than 50%, 40%, 35%, 33%, or 32%). For example, daily doses sufficient to provide substantial therapeutic effects in hypogonadal males e.g., serum (8R,9S,10R,13S,14S,17S)-17-Hydroxy-10,13-dimethyl-1,2,6,7,8,9,11,12,14,15,16,17-dodecahydrocyclopenta[a]phenanthren-3-one levels (e.g., C_{avg}) of greater than 300 ng/dL, can be achieved by the administration of 1, 2, 3, 4, 5, or 6 unit dosage forms as described herein. The unit dosage forms described herein can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg. The formulations also are release profile stable and have a suitable release profile (e.g., at least 50% in two hours). The formulations may also include an optional additive as described below in the following paragraphs. In one aspect, the composition is formulated as a capsule e.g., soft gel or hard gel.

The composition or dosage form as described herein can optionally include any of the following additives (some of which as the skilled artisan recognizes will act as the stabilizing agent or additive that allows for the production of compositions having the beneficial properties described herein) in the following seven paragraphs as long as the properties of the composition or dosage form are consistent with those as described herein.

Suitable additives utilized in various embodiments described herein include, by way of non-limiting example, adsorbing agents, anti-adherents, anticoagulants, antifoaming agents, antioxidants, anti-caking agents, anti-static agents, binders, bile acids, bufferants, bulking agents, chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agent sweeteners, stabilizers, surface area enhancing agents, suspending agent, thickeners, viscosity increasing agents, waxes and mixtures thereof.

Some non-limiting examples of the additives suitable for the present disclosure may be:

alcohols and/or polyols (e.g., ethanol, isopropanol, butanol, benzyl alcohol, ethylene glycol, propylene glycol, glycerol, sorbitol, mannitol, dimethyl isosorbide, polyethylene glycol, fatty acid alcohol, vinyl alcohol polypropylene glycol, polyvinyl alcohol, tocopherols, cellulose cyclodextrins, other derivatives, forms, mixtures thereof, or the like); ethers of polyethylene glycols having an average molecular weight of about 200 to about 20,000 (e.g., tetrahydrofurfuryl alcohol PEG ether, methoxy PEG, or the like); amides (e.g., 2-pyrrolidone, 2-piperidone, 8-caprolactam, N-alkylpyrrolidone, N-hydroxyalkylpyrrolidone, N-alkylpiperidone, N-alkylcaprolactam, dimethylacetamide, polyvinylpyrrolidone and the like.); esters (e.g., ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, 8-caprolactone and isomers thereof, 6-valerolactone and isomers thereof, gamma-butyrolactone and isomers thereof; and other additives known in the art, such as dimethyl acetamide, dimethyl isosorbide, N-methylpyrrolidones, monooctanoin, diethylene glycol monoethyl ether, or the like); amino acids (e.g., p-aminobenzamidine, sodium glycocholate) mesylate; amino acids and modified amino acids (e.g., aminoboronic acid derivatives and n-acetylcysteine; peptides and modified peptides (e.g., bacitracin, phosphinic acid dipeptide derivatives, pepstatin, antipain, leupeptin, chymostatin, elastin, bestatin, phoshporamindon, puromycin, cytochalasin potatocarboxy peptidase inhibitor, amastatin, or the like); polypeptide protease inhibitors; mucoadhesive polymers (e.g., polyacrylate derivatives, chitosan, cellulosics, chitosan-EDTA, chitosan-EDTA-antipain, polyacrylic acid, carboxymethyl cellulose etc.) or the like; or combinations thereof.

Some more examples of suitable additives for compositions and/or dosage forms described herein include, by way of non-limiting example, talc, magnesium stearate, silica (e.g., fumed silica, micronized silica, magnesium aluminum silicate etc.) and/or derivatives, polyethylene glycols, surfactants, waxes, oils, cetyl alcohol, polyvinyl alcohol, stearic acid, stearic acid salts, stearic acid derivatives, starch, hydrogenated vegetable oils, hydrogenated castor oils, sodium benzoate, sodium acetate, leucine, PEG, alkyl sulfate salts; acetylated monoglycerides; long-chain alcohols; silicone derivatives; butylated hydroxy toluene (BHT), butylated hydroxyl anisole (BHA), gallic acid, propyl gallate, ascorbic acid, ascorbyl palmitate, 4-hydroxymethyl-2,6-di-tert-butyl phenol, dry starch, dry sugars, polyvinyl pyrrolidones, starch paste, methacrylic copolymers, bentonite, sucrose, polymeric cellulose derivatives, shellac, sugar syrup; corn syrup; polysaccharides, acacia, tragacanth, guar gum, xanthan gums; alginates; gelatin; gelatin hydrolysate; agar; sucrose; dextrose; PEG, vinyl pyrrolidone copolymers, poloxamers; pregelatinized starch, sorbitol, glucose); acetic acid, hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, vinegar, pharmaceutically acceptable bases, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamin; salt of a pharmaceutically acceptable cation and an anion; EDTA and EDTA salts; titanium dioxide, food dyes, lakes, natural vegetable colorants, iron oxides, silicates, sulfates, magnesium hydroxide and aluminum hydroxide; halogenated hydrocarbons, trichloroethane, trichloroethylene, dichloromethane, fluorotrichloromethane, diethylether, trehalose, phosphates, citric acid, tartaric acid, gelatin, dextran and mannitol, lactose, mannitol, sodium chloride, potassium chloride, spray-dried lactose, hydrolyzed starches, directly compressible starch, microcrystalline cellulose, cellulosic derivatives, sorbitol, sucrose, sucrose-based materials, calcium sulfate, dibasic calcium phosphate, dextrose, croscarmellose sodium, starch, starch derivatives, clays, gums, cellulose, cellulose derivatives, alginates, crosslinked polyvinylpyrrolidone, sodium starch glycolate and microcrystalline cellulose, magnesium oxide, magnesium carbonates; desensitizers, spray-dried flavors, essential oils, ethyl vanillin, styrene/divinyl benzene copolymers, quaternary ammonium compounds, polyethylene glycol, citrate esters (such as triethyl citrate, acetyl triethyl citrate, acetyl tributyl citrate), acetylated monoglycerides, glycerin, triacetin, propylene glycol, phthalate esters (e.g., diethyl phthalate, dibutyl phthalate), castor oil, sorbitol and dibutyl sebacate, ascorbic acid, boric acid, sorbic acid, benzoic acid, and salts thereof, parabens, phenols, benzyl alcohol, and quaternary ammonium compounds; alcohols, ketones, esters, chlorinated hydrocarbons water; sweeteners (e.g., maltose, sucrose, glucose, sorbitol, glycerin and dextrins, aspartame, saccharine, saccharine salts, glycyrrhizin), viscosity modifiers, sugars, polyvinylpyrrolidone, cellulosics, polymers, gums and/or alginates.

In one embodiment, additives may also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum Arabic); spermaceti; natural or synthetic waxes; carnauba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based polymers (e.g., ethyl cellulose, methyl cellulose, microcrystalline cellulose, sodium carboxymethyl cellulose, hydroxypropylmethyl cellulose, ethyl cellulose, hydroxypropyl cellulose, HPMC acid succinates, cellulose acetate, cellulose nitrate, cellulose acetate butyrate, cellulose acetate trimellitate, carboxymethylethyl cellulose, hydroxypropylmethyl cellulose phthalate), shellacs; inorganics, such as dicalcium phosphate, hydroxyapatite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar. Non-limiting examples of compounds (e.g., additives) that can be used as at least a part of the pharmaceutically acceptable carrier include without limitation celluloses; dextrins, gums, carbomers, methacrylates, sugars, lactoses, inorganic carbonates, oxides, chlorides, sulphates and the like; salts of calcium; salts of magnesium; salts of fatty acids; inorganic and organic acids, bases and salts; propylene glycol; glycerols; fatty acids; fatty alcohols; fatty acid esters; glycerol esters; mono-, di- or triglycerides; edible oils; omega oils; vegetable oils, hydrogenated vegetable oils; partially or fully hydrogenated vegetable oils; glycerol esters of fatty acids; waxes; alcohols; gelatin; polyethylene glycol; polyethylene oxide co-polymers; silicates; antioxidants, tocopherols, sugar stearates, starches, shellac, resins, proteins, acrylates; methyl copolymers; polyvinyl alcohol; starch; phthalates; and combinations thereof.

In one embodiment, the additive may include at least one component selected from celluloses, dextrins, gums, carbomers, methacrylates, inorganic carbonates, salts of calcium, salts of magnesium, fatty acids, fatty acid esters, gelatin, lactoses, polyethylene glycol, polyethylene oxide co-polymers, silicates, partially hydrogenated vegetable oils, fully hydrogenated vegetable oils, waxes, antioxidants, tocopherol, sugar stearates, starches, shellac, resins, proteins, and combinations thereof.

In another embodiment, the additive may include at least one component selected from celluloses, dextrins, gums, carbomers, methacrylates, sugars, lactoses, inorganic carbonates, salts of calcium, salts of magnesium, salts of fatty acids, inorganic and organic acids, bases and salts, propylene glycol, glycerols, fatty acids, fatty alcohols, fatty acid esters, glycerol esters, mono-glycerol esters of fatty acids, di-glycerol esters of fatty acids, mixtures of mono-glycerol and di-gylcerol esters of fatty acids, omega oils, waxes, alcohols, gelatin, polyethylene glycol, polyethylene oxide co-polymers, silicates, antioxidants, tocopherol, sugar stearates, starches, shellac, resins, proteins, acrylates, methyl copolymers, polyvinyl alcohol, starch, phthalates, and combinations thereof.

Non-limiting examples of additives as release modulators that may be used include lipophilic resins; ethyl cellulose (EC), methylethyl cellulose (MEC), carboxymethyl ethylcellulose (CMEC), hydroxyethyl cellulose (HEC), cellulose acetate (CA), cellulose propionate (CPr), cellulose butyrate (CB), cellulose acetate butyrate (CAB), cellulose acetate phthalate (CAP), cellulose acetate trimellitate (CAT), hydroxypropyl methyl cellulose phthalate (HPMCP), hydroxypropyl methyl cellulose acetate succinate (HPMCAS), hydroxypropyl methyl cellulose acetate trimellitate (HPMCAT), ion-exchange resin; poloxamers; and ethylhydroxy ethylcellulose (EHEC) tocopherol; shellac; and combinations thereof. Non-limiting examples of lipidic lipophilic release modulators include fatty acids; mono-, di-, tri-esters of fatty acids with glycerol; sucrose esters with fatty acids; cetyl alcohol; stearic acid; glyceryl monostearate; glyceryl distearate; glyceryl tristearate; glyceryl palmitostearate; hydrogenated castor oil; butyl and glycol esters of fatty acids; oleic acid; cetyl alcohol; stearyl alcohol; cetostearyl alcohol; hydrogenated vegetable oil; waxes; bees wax; lard; omega fatty acid esters; hydrogenated soybean oil; hydrogenated vegetable oil; hydrogenated cottonseed and castor oil; partially hydrogenated soybean oil; partially hydrogenated castor oil; partially soy and cottonseed oil; phospholipids; hydrogenated oils, and their derivatives and combinations thereof.

In some embodiments, the composition described herein comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate (at greater than 23%, 24%, 25%, 26%, 27%, 28%, or 29% w/w drug loading (and less than 50%, 40%, 35%, 33%, or 32%)) and one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—CH₂)ₙ—OH where n is an integer from 3 to 900 wherein the composition when tested is a USP type 2 paddle apparatus having 1000 mL of 8% Octoxynol-9 (Triton-X100) in water at 37° C. (±0.5) (a) releases 80% or more of the active pharmaceutical ingredient at 12, 11, 10, 9, 8, 7, 6, 5, 4, 3, 2, 1, 0.5, or 0.25 hours (b) releases less than 100% at 12, 11, 10, 9, 8, 7, 6, 5, 4, 3, 2, 1, 0.5, or 0.25 hours (c) releases about 100% at 12, 11, 10, 9, 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, 2, 3, or 4 hours; (b) less than 100% at 6, 5, 4, 3, 2, 1, 0.5, or 0.25 hours; (c) about 100% at 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, or 2 hours; (b) less than 100% at 3, 2, 1, 0.5, or 0.25 hours; (c) about 100% at 4, 3, 2, 1, 0.5, or 0.25 hours or (d) a combination of one, two, or three of (a)-(c). In one aspect, the composition of this embodiment releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c). In certain aspects of this embodiment, the composition comprises from about 23% to about 35% 8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate active pharmaceutical ingredient and from 20% to 77% of one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; H—(O—CH₂—CH₂)ₙ—OH where n is an integer from 3 to 900; a branched; star, or comb analog of H—(O—CH₂—CH₂)ₙ—OH where n is an integer from 3 to 900. In a specific aspect, of this paragraph, the composition comprises 23%-35% (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate active pharmaceutical ingredient and 20%-77% one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; H—(O—CH₂—CH₂)ₙ—OH where n is an integer from 3 to 900; a branched; star, or comb analog of H—(O—CH₂—CH₂)ₙ—OH where n is an integer from 3 to 900; polyoxyethylated oil; polyoxyethylated hydrogenated vegetable oil; polyoxyethylated hydrogenated vegetable oil; and polyoxyethylated hydrogenated castor oil. In another specific aspect, the composition comprises 23%-35% of the active pharmaceutical ingredient and 20%-77% one or more components chosen from the following a C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid. In another specific aspect, the composition comprises 23%-35% lipophilic tetracyclic active pharmaceutical ingredient and 20%-77% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 23%-35% the active pharmaceutical ingredient and 20%-77% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C4 to C20 fatty acid. In another specific aspect, the composition comprises 25%-35% of the active pharmaceutical ingredient and 25%-75% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 25%-35% the active pharmaceutical ingredient and 30%-75% one or more components chosen from the following a C14 to C20 fatty acid; a mono-, di-, tri-propane-1,2,3-triol ester of a C14to C20 fatty acid; a combination (e.g., mixture) of mono-, di-, tri-propane-1,2,3-triol esters of a C14 to C20 fatty acid. In another specific aspect, the composition comprises 25%-35% of the active pharmaceutical ingredient and 40%-75% one or more components chosen from the following a C14 to C20 fatty acid; a mono-, di-, tri-propane-1,2,3-triol ester of a C14 to C20 fatty acid; a combination (e.g., mixture) of mono-, di-, tri-propane-1,2,3-triol esters of a C14 to C20 fatty acid. In another specific aspect, the composition comprises 23%-35% of the active pharmaceutical ingredient and 20%-77% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 23%-35% of the active pharmaceutical ingredient and 25%-77% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 25%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 30%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 40%-75% one or more components chosen from the following a C16 to C18 fatty acid; a mono-, di-, tri-propane-1,2,3-triol ester of a C16 to C18 fatty acid; a combination (e.g., mixture) of mono-, di-, tri-propane-1,2,3-triol esters of a C16 to C18 fatty acid. In another specific aspect, the composition comprises 25%-35% of the active pharmaceutical ingredient and 40%-75% one or more components chosen from the following (A) octadecanoic acid, (9Z)-octadec-9-enoic acid, hexadecanoic or a combination thereof; (B) a mono-, di-, tri-propane-1,2,3-triol ester of (A); (C) a combination of mono-, di-, or tri-propane-1,2,3-triol esters of (A); and (D) combination of one or more of (A)-(C). According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 1% to 25%, 0% to 15%, 1% to 15%, 0% to 10%, 1% to 10%, 0% to 8%, 1% to 8%, less than 5%, less than 4%, less than 3%, or less than 2%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In one embodiment, the composition comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate (at greater than 23%, 24%, 25%, 26%, 27%, 28%, or 29% w/w drug loading (and less than 50%, 40%, 35%, 33%, or 32%)) and one or a combination of mono-, di-, or tri-fatty acid esters of propane-1,2,3-triol. In a specific aspect, the one or a combination of mono-, di-, or tri-fatty acid esters of propane-1,2,3-triol are esters of one or more of octadecanoic acid or hexadecanoic acid. In one aspect, one or a combination of mono-, di-, or tri-fatty acid esters of propane-1,2,3-triol is an additive that allows for loading of the API in the formulation above its solubility limit without substantially compromising release profile, release profile stability, bioavailability or a combination thereof. In one aspect, the total mono-ester content is from about 0% to about 50%. In one aspect, the total mono-ester content is from about 2% to about 50%. In one aspect, the total mono-ester content is from about 3% to about 40%. In one aspect, the total mono-ester content is from about 4% to about 35%. In one aspect, the total di-ester content is from about 0% to about 90%. In one aspect, the total di-ester content is from about 10% to about 90%. In one aspect, the total di-ester content is from about 20% to about 80%. In one aspect, the total di-ester content is from about 25% to about 75%. %. In one aspect, the total tri-ester content is from about 0% to about 90%. In one aspect, the total tri-ester content is from about 5% to about 80%. In one aspect, the total tri-ester content is from about 15% to about 70%. In one aspect, the total tri-ester content is from about 15% to about 60%. In one aspect, the total tri-ester content is from about 15% to about 50%. In one aspect, the ester content is from about 10% to about 90% octadecanoic acid. In one aspect, the ester content is from about 20% to about 80% octadecanoic acid. In one aspect, the ester content is from about 25% to about 75% octadecanoic acid. In one aspect, the ester content is from about 30% to about 70% octadecanoic acid. In one aspect, the ester content is from about 10% to about 90% hexadecanoic acid. In one aspect, the ester content is from about 20% to about 80% hexadecanoic acid. In one aspect, the ester content is from about 25% to about 75% hexadecanoic acid. In one aspect, the ester content is from about 30% to about 70% hexadecanoic acid. In one aspect, the ester content is from about 30% to about 70% hexadecanoic acid; about 30% to about 70% octadecanoic acid; mono-ester content is from about 4% to about 35%; di-ester content is from about 25% to about 75%; and tri-ester content is from about 15% to about 50%. In one aspect, the melting point of the combination of mono-, di-, or tri-fatty acid esters of propane-1,2,3-triol is in the range of from about 30° C. to 100° C. In another aspect, the melting point of the combination of mono-, di-, or tri-fatty acid esters of propane-1,2,3-triol is in the range of from about 35° C. to 90° C. In another aspect, the melting point of the combination of mono-, di-, or tri-fatty acid esters of propane-1,2,3-triol is in the range of from about 40° C. to 80° C. In another aspect, the melting point of the combination of mono-, di-, or tri-fatty acid esters of propane-1,2,3-triol is in the range of from about 40° C. to 70° C. In another aspect, the melting point of the combination of mono-, di-, or tri-fatty acid esters of propane-1,2,3-triol is in the range of from about 45° C. to 65° C. Thus, according to one aspect of this embodiment, a composition is provided that has from about 0.1% to about 25% w/w of a combination of mono-, di-, or tri-fatty acid esters of propane-1,2,3-triol; from about 10% to about 40% w/w active pharmaceutical ingredient; from about 20% to about 75% fatty acid; and optionally, one or more pharmaceutically acceptable excipients. According to another aspect of this embodiment, a composition is provided that has from about 1% to about 20% w/w of a combination of mono-, di-, or tri-fatty acid esters of propane-1,2,3-triol; from about 20% to about 40% w/w active pharmaceutical ingredient; from about 20% to about 75% fatty acid; and optionally, one or more pharmaceutically acceptable excipients. According to yet another aspect of this embodiment, a composition is provided that has from about 1% to about 20% w/w of a combination of mono-, di-, or tri-fatty acid esters of propane-1,2,3-triol ; from about 25% to about 40% w/w active pharmaceutical ingredient; from about 20% to about 75% fatty acid; and optionally, one or more pharmaceutically acceptable excipients. According to again another aspect of this embodiment, a composition is provided that has from about 1% to about 20% w/w of a combination of mono-, di-, or tri-fatty acid esters of propane-1,2,3-triol; from about 25% to about 35% w/w pharmaceutical ingredient; from about 30% to about 75% fatty acid; and optionally, one or more pharmaceutically acceptable excipients. In an alternative aspect, the fatty acid is octadecanoic acid, (9Z)-octadec-9-enoic acid, (9Z,12Z)-9,12-octadecadienoic acid or hexadecanoic acid. In another alternative aspect, the fatty acid is a C16-C18 fatty acid. According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 0% to 15%, 0% to 10% or 1% to about 8%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In some embodiments, the composition described herein has a compound of formula: H—(O—CH₂—CH₂)ₙ—OH where n is an integer from 5 to 2000 and comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate (at greater than 23%, 24%, 25%, 26%, 27%, 28%, or 29% w/w drug loading (and less than 50%, 40%, 35%, 33%, or 32%)). The compound of formula: H—(O—CH₂—CH₂)ₙ—OH cans serve as a stabilizing agent that prevents or inhibits crystallization of API, allows for increased loading of API without substantially compromising the release profile, release profile stability, bioavailability or a combination thereof. In some aspects of this embodiment, n is an integer from 9 to 1000. In some aspects of this embodiment, n is an integer from 9 to 1000. In some aspects of this embodiment, n is an integer from 9 to 500. In some aspects of this embodiment, n is an integer from 9 to 500. In some aspects, the compound of formula: H—(O—CH₂—CH₂)ₙ—OH is characterized as having an average molecular weight of about 100 to about 50,000 gram/mol. In some aspects, the compound of formula: H—(O—CH₂—CH₂)—OH is characterized as having an average molecular weight of about 200 to about 30,000 gram/mol. In some aspects, In some aspects, the melting point of from about 30° C. to about 100° C. is characterized as having an average molecular weight of about 300 to about 20,000 gram/mol. In some aspects, the compound of formula: H—(O—CH₂—CH₂)ₙ—OH is characterized as having an average molecular weight of about 400 to about 20,000 gram/mol. In some aspects, the compound of formula: H—(O—CH₂—CH₂)ₙ—OH is characterized as having an average molecular weight of about 600 to about 15,000 gram/mol. In some aspects, the compound of formula: H—(O—CH₂—CH₂)ₙ—OH has a melting point of from about 4° C. to about 150° C. In some aspects, the melting point of from about 10° C. to about 100° C. In some aspects, the compound of formula: H—(O—CH₂—CH₂)ₙ—OH has a melting point of from about 20° C. to about 100° C. In some aspects, the compound of formula: H—(O—CH₂—CH₂)ₙ—OH has a melting point of from about 25° C. to about 100° C. In some aspects, the compound of formula: H—(O—CH₂—CH₂)ₙ—OH has a melting point of from about 20° C. to about 70° C. In some aspects, the compound of formula: H—(O—CH₂—CH₂)ₙ—OH has a melting point of from about 25° C. to about 60° C. In some aspects, the compound of formula: H—(O—CH₂—CH₂)ₙ—OH has a melting point of from about 30° C. to about 60° C. Thus, according to one aspect of this embodiment, a composition is provided having 0.1% to about 30% of a compound of formula: H—(O—CH₂—CH₂)ₙ—OH where n is an integer from 5 to 2000; from about 20% to about 40% w/w active pharmaceutical ingredient; from about 20% to about 75% fatty acid (e.g., C16-C18), from about 0% to about 20% w/w of a mono-, di-, or tri-fatty acid esters of propane-1,2,3-triol or a combination thereof; and optionally, one or more pharmaceutically acceptable excipients. In a specific aspect, the fatty acid is a C16 to C18 fatty acid. In a specific aspect, the fatty acid is octadecanoic acid, (9Z)-octadec-9-enoic acid, or hexadecanoic acid. In a specific aspect, the mono-, di-, or tri-fatty acid esters of propane-1,2,3-triol or a combination thereof are esters of octadecanoic acid, (9Z)-octadec-9-enoic acid, or hexadecanoic acid. In a specific aspect, the compound of formula: H—(O—CH₂—CH₂)ₙ—OH has a molecular weight of about 800 to 12000 gram/mol. In one aspect of this embodiment the active pharmaceutical ingredient is present in the composition in an amount of from about 10% to about 40% w/w. In another aspect of this embodiment the active pharmaceutical ingredient is present in the composition in an amount of from about 20% to 40% w/w. In again another aspect of this embodiment, the active pharmaceutical ingredient is present in the composition in an amount of from about 25% to 35% w/w. According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 0% to 15%, 0% to 10% or 1% to about 8%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In some embodiments, the composition described herein comprises (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate (at greater than 23%, 24%, 25%, 26%, 27%, 28%, or 29% w/w drug loading (and less than 50%, 40%, 35%, 33%, or 32%)) and one or more of 2-isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof. Accordingly, in a specific aspect, the composition comprises 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, in an amount ranging from about 5% to about 40% (w/w). In another specific aspect, the pharmaceutical composition (e.g., unit dosage form, formulation, or pharmaceutical composition) comprises from about 5% to about 40% (w/w) of a composition having 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, where 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof, is at least 10%, 20%, or 25% (1R,2S,5R)-2-isopropyl-5-methylcyclohexanol and at least 5%, 8%, or 12% (2S,5R)-2-Isopropyl-5-methylcyclohexanone. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In one embodiment, the (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate and is present in the composition in an amount of from about 20% to 40% w/w. In again another aspect of this embodiment, the API is present in the composition in an amount of from about 25% to 35% w/w. According to this embodiment, in some aspects, the composition can further comprise a polyoxyethylated oil; a polyoxyethylated hydrogenated vegetable oil; a polyoxyethylated hydrogenated oil; a polyoxyethylated hydrogenated castor oil; or a combination thereof and is present in an amount (w/w) of from 0% to 25%, 0% to 15%, 0% to 10% or 1% to about 8%. The composition can be formulated as a unit dosage form described herein and can have 150 mg or more, 200 mg or more, 225 mg or more, 250 mg or more, 275 mg or more, 300 mg or more, 325 mg or more, 350 mg or more, 375 mg or more, 400 mg or more, 425 mg or more, 450 mg or more, 475 mg or more, 500 mg or more, 525 mg or more, 550 mg or more, 575 mg or more, or 600 mg of API. The formulations also are release profile stable. The formulations may also include an optional additive as described above. In one aspect, the composition is formulated as a capsule e.g., soft gel or hard gel.

In one embodiment, the composition has, on a weight to weight basis, from about 20% to about 50%, 23% to 35%, or 26% to 32% of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and about 10% to about 90% of (a) one or more pharmaceutically acceptable excipients. According to this embodiment, the pharmaceutically acceptable excipients and amounts of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate are selected such that they allow for once or twice daily dosing of one, two, or three unit dosage forms to provide C_{avg} serum testosterone values in hypogonadal males of about 300 ng/dL or more, 350ng/dL or more, or 450 ng/dL or more. In some aspects of this embodiment, the composition is formulated such that C_{avg} values are at least 500 ng/dL, 550 ng/dL, 600 ng/dL or 650 ng/dL. In some aspects of this embodiment, the composition is formulated such that it minimizes supraphysiological serum testosterone levels. In a specific aspect, minimizes supraphysiological serum testosterone levels refers to greater than 2500 ng/dL in 1% or less of a population of patients, greater than 1800 ng/dL and less than 2500 ng/dL in less than 5% of a population of patients, or less than 1500 ng/dL in greater than 85% of a population of patients. As used herein a population can refer to 10-20, 20-50, or 50-100 individuals. In a specific aspect of this embodiment, the composition is formulated as a unit dosage forms and has greater than about 150 mg of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate. In another specific aspect, the composition is formulated as a unit dosage form and has greater than about 160 mg, 170 mg, 160 mg, 180 mg, 190 mg, 200 mg, 210 mg, 220 mg, 230 mg, 240 mg, 250 mg, 260 mg, 270 mg, 280 mg, 290 mg or 300 mg of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate. In another specific aspect, the composition is formulated as a unit dosage form and has less than about 600 mg, 500 mg, 475 mg, 450 mg, 440 mg, 430 mg, 420 mg, 410 mg, 400 mg, 390 mg, 380 mg, 370 mg, 360 mg, 350 mg, 340 mg or 330 mg of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate. In another specific aspect, the composition is formulated as a unit dosage form and has greater than about 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 360 mg, 380 mg, 390 mg, 400 mg, 410 mg, 420 mg, 430 mg, 440 mg, 450 mg, 460 mg, 470 mg, 480 mg, 490 mg or 500 mg of pharmaceutically acceptable excipient(s). In another specific aspect, the composition is formulated as a unit dosage form and has less than about 1000 mg, 950 mg, 900 mg, 850 mg, 800 mg, 750 mg, 700 mg, 650 mg, 600 mg, or 550 mg of pharmaceutically acceptable excipients. In one aspect of this embodiment, the composition is not a liquid at 5° C.; a gel, paste, or semi-solid at 15° C.; a liquid at 25, 30, 35 or 40° C.; or a combination thereof. In one aspect of this embodiment, one or more of the pharmaceutically acceptable excipients are (a) octadecanoic acid, (9Z)-octadec-9-enoic acid or hexadecanoic acid or (b) a mono-, di-, tri-propane-1,2,3-triol ester thereof, a combination of mono-, di-, or tri-propane-1,2,3-triol esters thereof or (c) any combination thereof. In some aspects, the pharmaceutically acceptable excipient may also be selected from or include 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; 2-isopropyl-5-methylcyclohexanol; or a combination thereof. In another aspect, the pharmaceutically acceptable excipient may also be chosen from a polyoxyethylated oil; a polyoxyethylated hydrogenated vegetable oil; a polyoxyethylated hydrogenated castor oil; a compound of formula H—(O—CH₂—CH₂)ₙ—OH where n is an integer from 5 to 600; or a combination thereof. The formulations also are release profile stable. The formulations may also include an optional additive as described above. In one aspect, the composition is formulated as a capsule e.g., soft gel or hard gel.

In another embodiment, the composition has, on a weight to weight basis, from about 20% to about 50%, 23% to 35%, or 26% to 32% of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and about 10% to about 90% of (a) octadecanoic acid, (9Z)-octadec-9-enoic acid or hexadecanoic acid (b) a mono-, di-, tri-propane-1,2,3-triol ester thereof, a combination of mono-, di-, or tri-propane-1,2,3-triol esters thereof or (c) a combination thereof. The formulations also are release profile stable. The formulations may also include an optional additive as described above. In one aspect, the composition is formulated as a capsule e.g., soft gel or hard gel.

In some embodiments, the composition described herein when tested with a USP type 2 paddle apparatus having 1000 mL of 8% Octoxynol-9 (Triton-X100) in water at 37° C. (±0.5) (a) releases 80% or more of the active pharmaceutical ingredient at 12, 11, 10, 9, 8, 7, 6, 5, 4, 3, 2, 1, 0.5, or 0.25 hours (b) releases less than 100% at 12, 11, 10, 9, 8, 7, 6, 5, 4, 3, 2, 1, 0.5, or 0.25 hours (c) releases about 100% at 12, 11, 10, 9, 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, 2, 3, or 4 hours; (b) less than 100% at 6, 5, 4, 3, 2, 1, 0.5, or 0.25 hours; (c) about 100% at 8, 7, 6, 5, 4, 3, 2, 1, 0.5, or 0.25 hours or (d) a combination of one, two, or three of (a)-(c). In one aspect, the composition of this embodiment releases (a) at least 80% or more at 0.25, 0.5, 1, or 2 hours; (b) less than 100% at 3, 2, 1, 0.5, or 0.25 hours; (c) about 100% at 4, 3, 2, 1, 0.5, or 0.25 hours or (d) a combination of one, two, or three of (a)-(c). In one aspect, the composition of this embodiment releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c). In certain aspects of this embodiment, the composition comprises from about 23% to about 35%, 24% to 35%, 25% to 33% or 26% to 32% (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and from 20% to 77% of one or more components chosen from the following: A C8 to C22 fatty acid; a mono-, di-, tri-propane-1,2,3-triol ester of a C8 to C22 fatty acid; a combination (e.g., mixture) of mono-, di-, tri-propane-1,2,3-triol esters of a C8 to C22 fatty acid; 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester;

(1R,2S,5R)-2-isopropyl-5-methylcyclohexanol; (2-isopropyl-5-methylcyclohexanol; polyoxyethylated oil; polyoxyethylated hydrogenated vegetable oil; polyoxyethylated hydrogenated vegetable oil; polyoxyethylated hydrogenated castor oil; or H—(O—CH₂—CH₂)ₙ—OH where n is an integer from 3 to 900. The formulations may also include an optional additive as described above. In one aspect, the composition is formulated as a capsule e.g., soft gel or hard gel. In one aspect, the composition has, on a weight to weight basis, from about 20% to about 50%, 23% to 35%, or 26% to 32% of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate.

### Exemplary Soft Gel Capsule Formulations

Provided in this section are exemplary soft gel capsules or soft gel capsule fill formulations having from about 20% to about 50%, 23% to 35%, or 26% to 32%, an active pharmaceutical ingredient which is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate and a pharmaceutically acceptable carrier which is a carrier for the active pharmaceutical ingredient. More specifically, the carrier is a solvent for the active pharmaceutical ingredient. Typically, these formulations are non-solid at room temperature. Desirably, the carrier allows for high concentrations of the active pharmaceutical ingredient without substantially compromising bioavailability. In some specific aspects, the carrier also includes one or more pharmaceutically acceptable additives. In one aspect, the carrier is a liquid at 4020 C. or more. In another aspect, the carrier is a liquid at 3820 C. or more. In yet another aspect, the carrier is a liquid at 3620 C. or more. In yet another aspect, the carrier is a liquid at 3420 C. or more. In yet another aspect, the carrier is a liquid at 3220 C. or more.

In yet another aspect, the carrier is a liquid at 3020 C. or more. In yet another aspect, the carrier is a liquid at 2720 C. or more. In yet another aspect, the carrier is a liquid at 2420 C. or more. In yet another aspect, the carrier is a liquid at 2020 C. or more. In yet another aspect, the carrier is a liquid at 1620 C. or more. In yet another aspect, the carrier is a liquid at 1220 C. or more. In one aspect of this embodiment, the active pharmaceutical ingredient has a solubility of greater than 5 mg/mL in the carrier at a temperature of 30-4020 C. In one aspect of this embodiment, the active pharmaceutical ingredient has a solubility of greater than 25 mg/mL in the carrier at a temperatureof 30-4020 C. In one aspect of this embodiment, the active pharmaceutical ingredient has a solubility of greater than 50 mg/mL in the carrier at a temperature of 30-4020 C. In one aspect of this embodiment, the active pharmaceutical ingredient has a solubility of greater than 75 mg/mL in the carrier at a temperature of 30-4020 C. In one aspect of this embodiment, testosterone tridecanoate has a solubility of greater than 100 mg/mL in the carrier at a temperature of 30-4020 C. In one aspect of this embodiment, the active pharmaceutical ingredient has a solubility of greater than 130 mg/mL in the carrier at a temperature of 30-4020 C. In one aspect of this embodiment, the active pharmaceutical ingredient has a solubility of greater than 160 mg/mL C in the carrier at a temperature of 30-4020 . In one aspect of this embodiment, the active pharmaceutical ingredient has a solubility of greater than 190 mg/mL in the carrier at a temperature of 30-4020 C. In one aspect of this embodiment, the active pharmaceutical ingredient has a solubility of greater than 220 mg/mL in the carrier at a temperature of 30-4020 C. In one aspect of this embodiment, the active pharmaceutical ingredient has a solubility of greater than 230 mg/mL in the carrier at a temperature of 30-4020 C. In one aspect of this embodiment, the active pharmaceutical ingredient has a solubility of greater than 240 mg/mL in the carrier at a temperature 30-4020 C. In one aspect of this embodiment, the active pharmaceutical ingredient has a solubility of greater than 250 mg/mL in the carrier at a temperature of 30-4020 C. In one aspect of this embodiment, the active pharmaceutical ingredient has a solubility of greater than 260 mg/mL in the carrier at a temperature of 30-4020 C. In one aspect of this embodiment, the active pharmaceutical ingredient has a solubility of greater than 270 mg/mL in the carrier at a temperature of 30-4020 C. In one aspect of this embodiment, the active pharmaceutical ingredient has a solubility of greater than 280 mg/mL in the carrier at a temperature of 30-4020 C. In one aspect of this embodiment, the active pharmaceutical ingredient has a solubility of greater than 290 mg/mL in the carrier at a temperature of 30-4020 C. In one aspect of this embodiment, the active pharmaceutical ingredient has a solubility of greater than 300 mg/mL in the carrier ata temperature of 30-4020 C. Typically, the solubility of the active pharmaceutical ingredient will be less than 600, 550, 500, 450, 400, 350 or 300 mg/mL. In one aspect of this embodiment, the carrier has 50% or less triglyceride. In one aspect of this embodiment, the carrier has 40% or less triglyceride. In one aspect of this embodiment, the carrier has 30% or less triglyceride. In one aspect of this embodiment, the carrier has 20% or less triglyceride. In one aspect of this embodiment, the carrier has 10% or less triglyceride. In one aspect of this embodiment, the carrier has 5% or less triglyceride. In one aspect of this embodiment, the carrier has 3% or less triglyceride. In one aspect of this embodiment, the carrier has 1% or less triglyceride. In one aspect of this embodiment, the carrier is substantially free of added triglyceride. In this context, substantially free of added triglyceride allows a level of triglyceride that is present as a minor or trace amount of another carrier that is used in the composition. For example, some monoglyceride carriers may also have an amount of triglyceride also as a component. Such a monoglyceride carrier if used in the composition described herein would be substantially free of added triglyceride. Conversely, if this same monoglyceride is used and a triglyceride is added to the carrier e.g, castor oil, this carrier is not considered substantially free of added triglyceride. In one aspect of this embodiment, one or more of the pharmaceutically acceptable carrier or excipients are (a) octadecanoic acid, (9Z)-octadec-9-enoic acid, hexadecanoic acid or (9Z,12Z)-9,12-Octadecadienoic acid (b) a mono-, di-, tri-propane-1,2,3-triol ester thereof, a combination of mono-, di-, or tri-propane-1,2,3-triol esters thereof or (c) any combination thereof. In one aspect, the pharmaceutically acceptable excipients or carriers can include polyethylene glycol, mono- or di-glyceride of stearic acid, palmitic acid, or a combination thereof, polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, or hydroxypropylmethyl cellulose acetate succinate. In one aspect, the composition of this embodiment releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c).

In one embodiment, the pharmaceutical composition or dosage form can have an additive. According to this embodiment, the additive can be any pharmaceutically acceptable additive. In a specific aspect, the pharmaceutical acceptable additive has the function of providing the (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate in a form that provides sufficient bioavailability to a subject when administered orally. In one aspect, the additive allows for loading of the API in the carrier at levels above the solubility of the API in the carrier while not substantially compromising properties of the formulation. For example, in one aspect, the additive prevents or reduces or inhibits the amount of crystallization of the (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. In one aspect, the additive increases the viscosity of the composition. In another aspect, the additive provides dissolution stability. In yet another aspect, the additive prevents or reduces degradation of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. In one aspect, the compositions have from about 20% to about 50%, 23% to 35%, or 26% to 32% of API. In a specific aspect, the additive is a polyethylene glycol. In a specific aspect, the polyethylene glycol has an average molecular weight of less 1000. In another specific aspect, the polyethylene glycol has an average molecular weight of less than 800. In yet another specific aspect, the polyethylene glycol has an average molecular weight of less than 500. In one aspect, the polyethylene glycol has a melting point of less than 5520 C. In one aspect, the polyethylene glycol has a melting point of less than 4520 C. In one aspect, the polyethylene glycol has a melting point of less than 3520 C. In one aspect, the polyethylene glycol has a melting point of less than 2520 C. In one aspect, the polyethylene glycol has a melting point of less than 1520 C. In one aspect, the polyethylene glycol has a melting point of less than 1020 C. In another aspect, the additive is a mono- or di-glyceride of stearic acid, palmitic acid, or a combination thereof. In one aspect, the additive is polvinylpyrrolidone. In one aspect, the additive is hydroxypropyl methylcellulose. In one aspect, the additive is hydroxypropyl cellulose. In one aspect, the additive is cellulose acetate phthalate. In one aspect, the additive is polyvinyl acetate phthalate. In one aspect, the additive is polyethylene oxide. In one aspect, the additive is poly(acrylic acid). In one aspect, the additive is polymethyacrylate. In one aspect, the additive is poly(ethylene oxide)-poly(propylene oxide)-poly(ethylene oxide). In one aspect, the additive is polyvinyl alcohol. In one aspect, the additive is polystyrenesulfonic acid. In one aspect, the additive is polyvinylpyrrolidone-co-polyvinyl acetate. In one aspect, the additive is polyether polyol. In one aspect, the additive is carboxymethylcellulose. In one aspect, the additive is methylcellulose. In one aspect, the additive is hydroxyethyl cellulose. In one aspect, the additive is hydroxypropylmethyl cellulose phthalate. In one aspect, the additive is hydroxypropylmethyl cellulose acetate succinate. In one aspect, the composition of this embodiment releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c).

### Exemplary Hard Gel Capsule Formulations

Provided in this section are exemplary hard gel capsule fill formulations having from about (in w/w) 20% to about 50%, 23% to 35%, or 26% to 32%, an active pharmaceutical ingredient which is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate and a pharmaceutically acceptable carrier which is a carrier for the active pharmaceutical ingredient. Typically, these formulations are non-liquid at room temperature. More specifically, the carrier is a solvent for the active pharmaceutical ingredient. Desirably, the carrier allows for high concentrations of the active pharmaceutical ingredient without substantially compromising bioavailability. In some specific aspects, the carrier also includes one or more pharmaceutically acceptable additives (e.g., stabilizing agent). In one aspect, the carrier is a liquid at 4020 C. or more. In another aspect, the carrier is a liquid at 3820 C. or more. In yet another aspect, the carrier is a liquid at 3620 C. or more. In yet another aspect, the carrier is a liquid at 3420 C. or more. In yet another aspect, the carrier is a liquid at 3220 C. or more. In yet another aspect, the carrier is a liquid at 3020 C. or more. In yet another aspect, the carrier is a liquid at 2720 C. or more. In yet another aspect, the carrier is a liquid at 2420 C. or more. In yet another aspect, the carrier is a liquid at 2020 C. or more. In yet another aspect, the carrier is a liquid at 1620 C. or more. In yet another aspect, the carrier is a liquid at 1220 C. or more. In one aspect of this embodiment, the active pharmaceutical ingredient has a solubility of greater than 5 mg/mL in the carrier at a temperature of 30-4020 C. In one aspect of this embodiment, the active pharmaceutical ingredient has a solubility of greater than 25 mg/mL in the carrier at a temperature of 30-4020 C. In one aspect of this embodiment, the active pharmaceutical ingredient has a solubility of greater than 50 mg/mL in the carrier at a temperature of 30-4020 C. In one aspect of this embodiment, the active pharmaceutical ingredient has a solubility of greater than 75 mg/mL in the carrier at a temperature of 30-4020 C. In one aspect of this embodiment, testosterone tridecanoate has a solubility of greater than 100 mg/mL in the carrier at a temperature of 30-4020 C. In one aspect of this embodiment, the active pharmaceutical ingredient has a solubility of greater than 130 mg/mL in the carrier at a temperature of 30-4020 C. In one aspect of this embodiment, the active pharmaceutical ingredient has a solubility of greater than 160 mg/mL C in the carrier at a temperature of 30-4020 . In one aspect of this embodiment, the active pharmaceutical ingredient has a solubility of greater than 190 mg/mL in the carrier at a temperature of 30-4020 C. In one aspect of this embodiment, the active pharmaceutical ingredient has a solubility of greater than 220 mg/mL in the carrier at a temperature of 30-4020 C. In one aspect of this embodiment, the active pharmaceutical ingredient has a solubility of greater than 230 mg/mL in the carrier at a temperature of 30-4020 C. In one aspect of this embodiment, the active pharmaceutical ingredient has a solubility of greater than 240 mg/mL in the carrier at a temperature 30-4020 C. In one aspect of this embodiment, the active pharmaceutical ingredient has a solubility of greater than 250 mg/mL in the carrier at a temperature of 30-4020 C. In one aspect of this embodiment, the active pharmaceutical ingredient has a solubility of greater than 260 mg/mL in the carrier at a temperature of 30-4020 C. In one aspect of this embodiment, the active pharmaceutical ingredient has a solubility of greater than 270 mg/mL in the carrier at a temperature of 30-4020 C. In one aspect of this embodiment, the active pharmaceutical ingredient has a solubility of greater than 280 mg/mL in the carrier at a temperature of 30-4020 C. In one aspect of this embodiment, the active pharmaceutical ingredient has a solubility of greater than 290 mg/mL in the carrier at a temperature of 30-4020 C. In one aspect of this embodiment, the active pharmaceutical ingredient has a solubility of greater than 300 mg/mL in the carrier at a temperature of 30-4020 C. Typically, the solubility of the active pharmaceutical ingredient will be less than 600 mg/mL. In one aspect of this embodiment, the carrier has 50% or less triglyceride. In one aspect of this embodiment, the carrier has 40% or less triglyceride. In one aspect of this embodiment, the carrier has 30% or less triglyceride. In one aspect of this embodiment, the carrier has 20% or less triglyceride. In one aspect of this embodiment, the carrier has 10% or less triglyceride. In one aspect of this embodiment, the carrier has 5% or less triglyceride. In one aspect of this embodiment, the carrier has 3% or less triglyceride. In one aspect of this embodiment, the carrier has 1% or less triglyceride. In one aspect of this embodiment, the carrier is substantially free of added triglyceride. In this context, substantially free of added triglyceride allows a level of triglyceride that is present as a minor or trace amount of another carrier that is used in the composition. For example, some monoglyceride carriers may also have an amount of triglyceride also as a component. Such a monoglyceride carrier if used in the composition described herein would be substantially free of added triglyceride. Conversely, if this same monoglyceride is used and a triglyceride is added to the carrier e.g, castor oil, this carrier is not considered substantially free of added triglyceride. In one aspect of this embodiment, one or more of the pharmaceutically acceptable carrier or excipients are (a) octadecanoic acid, (9Z)-octadec-9-enoic acid or hexadecanoic acid (b) a mono-, di-, tri-propane-1,2,3-triol ester thereof, a combination of mono-, di-, or tri-propane-1,2,3-triol esters thereof or (c) any combination thereof. In one aspect, the pharmaceutically acceptable excipients or carriers can include polyethylene glycol, mono- or di-glyceride of stearic acid, palmitic acid, or a combination thereof, polvinylpyrrolidone, hydroxypropyl methylcellulose, hydroxypropyl cellulose, cellulose acetate phthalate, polyvinyl acetate phthalate, polyethylene oxide, poly(acrylic acid), polymethyacrylate, poly(ethylene oxide)-poly(propylene oxide)-poly(ethylene oxide), polyvinyl alcohol, polystyrenesulfonic acid, polyvinylpyrrolidone-co-polyvinyl acetate, polyether polyol, carboxymethylcellulose, methylcellulose, hydroxyethyl cellulose, hydroxypropylmethyl cellulose phthalate, or hydroxypropylmethyl cellulose acetate succinate. In one aspect, the composition of this embodiment releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c).

### Exemplary Formulations Embodiments

Provided in this section are formulations.

TABLE 1
Drug + Carriers

| | Compositions (w/w %) | | Ratio of API:Carrier |
|---|---|---|---|
| | Testosterone | | in a pharmaceutical |
| Composition | tridecanoate* | Carrier | composition |
| A | 10-15 | 85-90 | 1:5.7-1:9.0 |
| B | 15-20 | 80-85 | 1:4.0-1:5.7 |
| C | 20-30 | 70-80 | 1:2.3-1:4.0 |
| D | 30-40 | 60-70 | 1:1.5-1:2.3 |
| E | 40-50 | 50-60 | 1:1.0-1:1.5 |

*As an active ingredient, it can be untreated, sieved (PS < 450 micron), milled (PS < 150 micron), micronized (1 micron < PS < 25 micron), or nanosized (PS < 1 micron).

TABLE 2
Carrier Components

| | | Carrier No. | | | | | | | | | | | | |
|---|---|---|---|---|---|---|---|---|---|---|---|---|---|---|
| Component | | I | II | III | IV | V | VI | VII | VIII | IX | X | XI | XII | XIII |
| Solubilizer | Propylene | Y | — | — | — | — | — | — | — | — | — | — | — | — |
| | glycol mono | | | | | | | | | | | | | |
| | or dilaurate | | | | | | | | | | | | | |
| | Propylene | — | Y | — | — | — | — | — | — | — | — | — | — | — |
| | glycol mono | | | | | | | | | | | | | |
| | or dicaprylate | | | | | | | | | | | | | |
| | Corn | — | — | Y | — | — | — | — | — | — | — | — | — | — |
| | glycerides | | | | | | | | | | | | | |
| | (e.g. Glyceryl | | | | | | | | | | | | | |
| | mono or dilinoleate) | | | | | | | | | | | | | |
| | Vegetable | — | — | — | Y | — | — | — | — | — | — | — | — | — |
| | glycerides | | | | | | | | | | | | | |
| | (e.g. Glyceryl | | | | | | | | | | | | | |
| | mono or dioleate) | | | | | | | | | | | | | |
| | Glyceryl mono | — | — | — | — | Y | — | — | — | — | — | — | — | — |
| | or distearate | | | | | | | | | | | | | |
| | Glyceryl | — | — | — | — | — | Y | — | — | — | — | — | — | — |
| | palmitostearate | | | | | | | | | | | | | |
| | (9Z)-Octadec- | — | — | — | — | — | — | Y | — | — | — | — | — | — |
| | 9-enoic acid | | | | | | | | | | | | | |
| | Octadecanoic | — | — | — | — | — | — | — | Y | — | — | — | — | — |
| | acid | | | | | | | | | | | | | |
| | (9Z,12Z)-9,12- | — | — | — | — | — | — | — | — | Y | — | — | — | — |
| | Octadecadienoic | | | | | | | | | | | | | |
| | acid | | | | | | | | | | | | | |
| | Peppermint oil | — | — | — | — | — | — | — | — | — | Y | — | — | — |
| | Omega-3 | — | — | — | — | — | — | — | — | — | — | Y | — | — |
| | EPA/DHA | | | | | | | | | | | | | |
| | Vitamin E | — | — | — | — | — | — | — | — | — | — | — | Y | — |
| | Combinations* | — | — | — | — | — | — | — | — | — | — | — | — | Y |
| Hydrophilic | Cremophor, | Y | Y | Y | Y | Y | Y | Y | Y | Y | Y | Y | Y | Y |
| Add. | Tween, SLS, | | | | | | | | | | | | | |
| | Poloxamer, | | | | | | | | | | | | | |
| | Polymer, | | | | | | | | | | | | | |
| | and/or | | | | | | | | | | | | | |
| | combinations | | | | | | | | | | | | | |
| Other | Antioxidant, | q.s. | q.s. | q.s. | q.s. | q.s. | q.s. | q.s. | q.s. | q.s. | q.s. | q.s. | q.s. | q.s. |
| Add. | solidifier, flow | | | | | | | | | | | | | |
| | agent, solvent, | | | | | | | | | | | | | |
| | and/or | | | | | | | | | | | | | |
| | combinations | | | | | | | | | | | | | |

*Combinations of solubilizers can be a combination of 2 or more solubilizers that are listed in this table as well as include propylene glycol, polyethylene glycol, glycerol, sorbitol, DMA, and so on.
Add. = additive.
Y = Yes.

### Carrier I. Compositions Composed of Solubilizer (Propylene Glycol Mono or Di-Laurate), Hydrophilic Additives, and Other Additives For Composition A to E

| | Carrier Compositions (w/w %) | | | | | | |
|---|---|---|---|---|---|---|---|
| | | Hydrophilic | | | | | % Release in |
| | | additives¹ (e.g. | | | | | 8% Triton |
| | | Cremophor² RH | | | | | Aqueous |
| | Propylene | 40, Tween³ 80, | | | | | Media |
| | glycol | SLS, Poloxamer⁴ | Other additives⁷ | | | | ≥50% in 2 hrs |
| Comp. | mono or | 407, Polymer⁵, | | Solidify | | Total % | and ≥75% in |
| I No. | dilaurate | and/or combinations⁶) | Antioxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Hydrophilic additives can be, but are not limited to ones listed in this table, e.g., hydrophilic surfactants having an HLB value of greater than 10, which are PEG-8 caprylic/capric glycerides (Labrasol), lauroyl macrogol-32 glyceride (Gelucire 44/14), stearoyl macrogol glyceride (Gelucire 50/13), sodium dioctyl sulfosuccinate, polyethylene glycol fatty acids mono- and di-ester mixtures, polyethylene glycol 1000 tocopherol succinate, phytosterols, phytosterol fatty acid esters, lanosterol PEG-24 cholesterol ether, PEG-30 soya sterol, PEG-25 phyto sterol, PEG-30 cholestanol, and so on.
² Cremophor includes, but is not limited to, Cremophor RH 40, but Cremophor EL, RH 40, and RH 60.
³ Tween includes, but is not not limited to, Tween 80, but Tween 20, 60, and 80.
⁴ Poloxamer includes, but is not limited to Poloxamer 407, but Poloxamer 124, 188, 234, 335, and 407.
⁵ Polymer includes, but is not limited to, Polyethylene glycol, Hydroxypropyl cellulose, Hydroxypropylmethyl cellulose, Hydroxypropylmethyl cellulose acetate succinate, Polyvinylpyrrolidone, Polyvinyl acetate, Polylactic-co-glycolic acid, Polyvinyl caprolactame, Carbomer, and a combination thereof.
⁶ Combinations of hydrophilic additives can be 2 or more hydrophilic additives.
⁷ Other additives can be, but are not not limited to ones listed in this table, e.g., adsorbing agents, anti-adherents, anticoagulants, antifoaming agents, anti-caking agents, anti-static agents, binders, bile acids, bufferants, bulking agents, chelating agents, coagulants, colorants, opaquants, coolants, cryoprotectants, diluents, dehumidifying agents, desiccants, desensitizers, disintegrants, dispersing agents, enzyme inhibitors, fillers, hydrating agent, super disintegrants, gums, mucilages, hydrogen bonding agents, enzymes, flavorants, humectants, humidifying agents, ion-exchange resins, lubricants, plasticizers, pH modifying agents, preservatives, organic solvents, spreading agent, stabilizers, suspending agent, thickeners, viscosity increasing agents, waxes, and so on.
⁸ Anti-oxidant can be, but is not limited to, ascorbyl palmitate, ascorbic acid, butylated hydroxyanisole (BHA), butylated hydroxytoluene (BHT), propyl gallate, cysteine, sodium metabisulfite (SMB), thiol derivatives, alpha-tocopherol, and so on.
⁹ Solidifying (solidify) agent can be, but is not limited, to PEG 3350, PEG 4000, PEG 6000, PEG 8000, Poloxamer 188, Poloxamer 407, cetyl esters, wax, beeswax, glyceryl monostearate, glyceryl distearate, glyceryl palmitostearate, stearic acid, and so on.
¹⁰ Combinations of other additives can be 2 or more other additives.
   Descriptions for from ¹ to ¹⁰ are applied to tables of all carrier compositions (from Carrier Ito Carrier XIII tables) shown below.

### Carrier II. Compositions Composed of Solubilizer (Propylene Glycol Mono or Di-Caprylate), Hydrophilic Additives, and Other Additives For Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | | Cremophor² RH | | | | | 8% Triton |
| | | 40, Tween³ 80, | | | | | Aqueous |
| | Propylene | SLS, Poloxamer⁴ | | | | | Media |
| | glycol | 407, Polymer⁵, | Other additives⁷ | | | | ≥50% in 2 hrs |
| Comp. | mono or | and/or | | Solidify | | Total % | and ≥75% in |
| II No. | dicaprylate | combinations⁶) | Antioxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier III. Compositions Composed of Solubilizer (Corn Glycerides: e.g. Glyceryl Mono or Di-Linoleate), Hydrophilic Additives, and Other Additives For Composition A to E

| | Carrier Compositions (w/w %) | | | | | | |
|---|---|---|---|---|---|---|---|
| | | Hydrophilic | | | | | |
| | | additives¹ (e.g. | | | | | % Release in |
| | Corn | Cremophor² RH | | | | | 8% Triton |
| | glycerides | 40, Tween³ 80, | | | | | Aqueous |
| | (e.g. | SLS, Poloxamer⁴ | | | | | Media |
| | Glyceryl | 407, Polymer⁵, | Other additives⁷ | | | | ≥50% in 2 hrs |
| Comp. | mono or | and/or | | Solidify | | Total % | and ≥75% in |
| III No. | dilinoleate) | combinations⁶) | Antioxidant⁸ | agent⁹ | Combinations¹⁰ | in Carrier | 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

Carrier IV. Compositions Composed of Solubilizer (Vegetable Glycerides: e.g. Glyceryl Mono or Di-Oleate), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | Vegetable | Cremophor² RH 40, | | | | | Aqueous |
| | glycerides | Tween³ 80, SLS, | | | | | Media |
| | (e.g. Glyceryl | Poloxamer⁴ 407, | Other additives⁷ | | | | ≥50% in 2 |
| Comp. | mono or | Polymer⁵, and/or | Anti- | Solidify | | Total % | hrs and ≥75% |
| IV No. | di-oleate) | combinations⁶)) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

### Carrier V. Compositions Composed of Solubilizer (Glyceryl Mono or Di-Stearate), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | Glyceryl | Poloxamer⁴ 407, | Other additives⁷ | | | | ≥50% in 2 |
| Comp. | mono or | Polymer⁵, and/or | Anti- | Solidify | | Total % | hrs and ≥75% |
| V No. | di-stearate | combinations⁶) | oxidant⁸ | agent⁹ | Combinations¹⁰ | in Carrier | in 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

### Carrier VI. Compositions Composed of Solubilizer (Glyceryl Palmito-Stearate), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | Glyceryl | Poloxamer⁴ 407, | Other additives⁷ | | | | ≥50% in 2 |
| Comp. | palmito- | Polymer⁵, and/or | Anti- | Solidify | | Total % | hrs and ≥75% |
| VI No. | stearate | combinations⁶) | oxidant⁸ | agent⁹ | Combinations¹⁰ | in Carrier | in 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

### Carrier VII. Compositions Composed of Solubilizer ((9Z)-Octadec-9-Enoic Acid), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | | Poloxamer⁴ 407, | Other additives⁷ | | | | ≥50% in 2 |
| Comp. | (9Z)-Octadec- | Polymer⁵, and/or | Anti- | Solidify | | Total % | hrs and ≥75% |
| VII No. | 9-enoic acid | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

### Carrier VIII. Compositions Composed of Solubilizer (Octadecanoic Acid), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | | Poloxamer⁴ 407, | Other additives⁷ | | | | ≥50% in 2 |
| Comp. | Octadecanoic | Polymer⁵, and/or | Anti- | Solidify | | Total % | hrs and ≥75% |
| VIII No. | acid | combinations⁶) | oxidant⁸ | agent⁹ | Combinations¹⁰ | in Carrier | in 4 hrs |
| a | 90-100 | — | 0-1 | 0-10 | 0-10 | 100 | No |
| b | 45-99 | 1-40 | 0-1 | 0-15 | 0-15 | 100 | No |
| | | 1-25 | 0-1 | 15-20 | 15-20 | 100 | No |
| c | 85-99 | 1-4.5 | 0-1 | 0-10 | 0-10 | 100 | No |
| d | 80-95 | 5-10 | 0-1 | 0-10 | 0-10 | 100 | No |
| e | 70-90 | 10-20 | 0-1 | 0-10 | 0-10 | 100 | No |
| f | 60-80 | 20-30 | 0-1 | 0-10 | 0-10 | 100 | No |
| | | 10-20 | 0-1 | 10-20 | 10-20 | 100 | No |
| g | 50-80 | 30-40 | 0-1 | 0-10 | 0-10 | 100 | No |
| | | 20-30 | 0-1 | 0-10 | 0-10 | 100 | No |
| | | 10-20 | 0-1 | 10-20 | 10-20 | 100 | No |

### Carrier IX. Compositions Composed of Solubilizer ((9Z,12Z)-9,12-Octadecadienoic Acid), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | (9Z,12Z)-9,12- | Poloxamer⁴ 407, | Other additives⁷ | | | | ≥50% in 2 |
| Comp. | Octadecadienoic | Polymer⁵, and/or | Anti- | Solidify | | Total % | hrs and ≥75% |
| IX No. | acid | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

### Carrier X. Compositions Composed of Solubilizer (Peppermint Oil), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | | Poloxamer⁴ 407, | Other additives⁷ | | | | ≥50% in 2 |
| Comp. | Peppermint | Polymer⁵, and/or | Anti- | Solidify | | Total % | hrs and ≥75% |
| X No. | oil | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | Yes |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | Yes |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | Yes |

### Carrier XI. Compositions Composed of Solubilizer (Omega-3 EPA/DHA), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | | Poloxamer⁴ 407, | Other additives⁷ | | | | ≥50% in 2 |
| Comp. | Omega-3 | Polymer⁵, and/or | Anti- | Solidify | | Total % | hrs and ≥75% |
| XI No. | EPA/DHA | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

### Carrier XII. Compositions Composed of Solubilizer (Vitamin E), Hydrophilic Additives, and Other Additives For Composition A to E

| | Carrier Compositions (w/w %) | | | | | | |
|---|---|---|---|---|---|---|---|
| | | Hydrophilic | | | | | % Release |
| | | additives¹ (e.g. | | | | | in 8% Triton |
| | | Cremophor² RH 40, | | | | | Aqueous |
| | | Tween³ 80, SLS, | | | | | Media |
| | | Poloxamer⁴ 407, | Other additives⁷ | | | | ≥50% in 2 |
| Comp. | | Polymer⁵, and/or | Anti- | Solidify | | Total % | hrs and ≥75% |
| XII No. | Vitamin E | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | Yes |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | Yes |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | Yes |

### Carrier XIII. Compositions Composed of a Combination of Solubilizers, Hydrophilic Additives, and Other Additives For Composition A to E

| | Carrier Compositions (w/w %) | | | | | | |
|---|---|---|---|---|---|---|---|
| | Combination | | | | | | |
| | of solubilizers^{†} | Hydrophilic | | | | | % Release |
| | (e.g. oleic acid | additives¹ (e.g. | | | | | in 8% Triton |
| | and GDS, | Cremophor² RH 40, | | | | | Aqueous |
| | oleic acid and | Tween³ 80, SLS, | | | | | Media |
| | peppermint oil, | Poloxamer⁴ 407, | Other additives⁷ | | | | ≥50% in 2 |
| Comp. | maisine 35-1 | Polymer⁵, and/or | Anti- | Solidifying | | Total % | hrs and ≥75% |
| XIII No. | and GMC, etc) | combinations⁶) | oxidant⁸ | agent⁹ | Combinations¹⁰ | in Carrier | in 4 hrs |
| a | 90-100 | — | 0-1 | 0-10 | 0-10 | 100 | Yes |
| b | 45-99 | 1-40 | 0-1 | 0-15 | 0-15 | 100 | Yes |
| | | 1-25 | 0-1 | 15-20 | 15-20 | 100 | No |
| c | 85-99 | 1-4.5 | 0-1 | 0-10 | 0-10 | 100 | Yes |
| d | 80-95 | 5-10 | 0-1 | 0-10 | 0-10 | 100 | Yes |
| e | 70-90 | 10-20 | 0-1 | 0-10 | 0-10 | 100 | Yes |
| f | 60-80 | 20-30 | 0-1 | 0-10 | 0-10 | 100 | Yes |
| | | 10-20 | 0-1 | 10-20 | 10-20 | 100 | No |
| g | 50-80 | 30-40 | 0-1 | 0-10 | 0-10 | 100 | Yes |
| | | 20-30 | 0-1 | 0-10 | 0-10 | 100 | Yes |
| | | 10-20 | 0-1 | 10-20 | 10-20 | 100 | No |

^{†}Combination of solubilizers includes, not limited to oleic acid and glyceryl distearate, oleic acid and peppermint oil, or maisine 35-1 and glyceryl monocaprylate, but 2 or more solubilizers that are listed in Table 2.

In one aspect, the composition of this embodiment in the Tables above releases (a) at least 80% or more at 1 or 2 hours; (b) less than 95% or 90% at 0.25 hours; (c) about 100% at 4, 3, 2, 1, 0.5, or 0.25 hours or (d) a combination of one, two, or three of (a)-(c) when tested using USP type 2 apparatus in about e.g., 1000 mL 8% Triton X100 solution in water at a specific temperature (e.g., 3720 C.) at 100 rpm Methods

Described herein, the inventors have discovered compositions, methods and dosing regimens that, in some embodiments, provide unexpectedly high serum C_{avg} testosterone levels while minimizing Cₘₐₓ values and mimic the natural diurnal peak to trough serum testosterone levels of eugonadal males. Without being bound by theory, it is believed that the provision of serum C_{avg} testosterone levels that are typically higher than those seen with other testosterone replacement therapies while minimizing supraphysiological excursions provides hypogonadal with clinical benefits. Moreover, the compositions and methods, in some aspects, have beneficial effects one or more of spermatogenesis, sperm motility, sperm quality, serum testosterone levels below a specified level (e.g., amount of time at trough), and Tₘₐₓ. The results described herein are even more exciting given that they were obtained using an oral route of administration, where pharmacokinetic variations between individuals seem to be more pronounced as compared to other routes of administration e.g., injection.

The inventive compositions, methods and dosing regimens provide surprising benefits over currently market testosterone replacement therapies by providing higher C_{avg} values while reducing the likelihood of Cₘₐₓ excursions. Thus, C_{avg} testosterone values of greater than 300 ng/dL, 350 ng/dL, 400 ng/dL45 0 ng/dL, 500 ng/dL, 550 ng/dL, 600 ng/dL, or 650 ng/dL can be achieved in a hypogonadal patient or a substantial portion of hypogonadal patients while Cₘₐₓ values remain in substantially safe range e.g., greater than 2500 ng/dL in 1% or less of a population of patients, greater than 1800 ng/dL and less than 2500 ng/dL in less than 5% of a population of patients, or less than 1500 ng/dL in greater than 85% of a population of patients.

Accordingly, a pharmaceutical composition is provided herein, having an amount of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate ranging from about 200 mg to about 500 mg and a pharmaceutically acceptable carrier, that is a unit dosage form for oral administration.

Furthermore, a method of administration of a testosterone replacement therapy is provided herein wherein the method involves administering one, two, three, four, five or six, unit dosage forms once daily. Additionally, a method of administration of a testosterone replacement therapy is provided herein wherein the method involves administering four or less unit dosage forms once daily. Furthermore, a method of administration of a testosterone replacement therapy is provided herein wherein the method involves administering three or less unit dosage forms once daily. Additionally, these administrations can be divided into twice daily or more administration.

Moreover, provided herein is a method of producing serum (8R,9S,10R,13S,14S,17S)-17-Hydroxy-10,13-dimethyl-1,2,6,7,8,9,11,12,14,15,16,17-dodecahydrocyclopenta[a]phenanthren-3-one daily patterns of a eugonadal male in a hypogonadal male by administering to a hypogondal male a sufficient amount of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate in a dosing regimen sufficient to mimic natural serum testosterone levels of a eugonadal male. This method provides daily fluctuations and levels of serum testosterone in hypogonadal males that are more similar to that of eugonadal males than any other approved testosterone replacement therapy that the inventors are aware of For example, eugonadal males (e.g., young 20-30 years old) have higher average serum concentrations of testosterone than older men and more fluctuation through the course of a day. In young healthy men, serum testosterone levels in peak in the morning and decline to a minimum at bed time. In older men, serum testosterone levels are more flat as compared to the fluctuations seen in young men. One method disclosed herein provides peak testosterone levels in the morning which decline until about bedtime. Morning peak serum testosterone levels are provided by administering one, two, or three unit dosage forms of a pharmaceutical composition having (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and a pharmaceutically acceptable carrier at a specified time. The amount of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate administered in combination with the pharmaceutically acceptable carrier provides maximum concentrations of serum testosterone at from about 6 AM to 2 PM and also provides C_{avg} serum testosterone levels that are in the eugonadal range of about 300 ng/dL to about 1100 ng/dL. Additional, in some aspects of this method, serum testosterone troughs occur about twelve hours after Cₘₐₓ. Thus, the inventors have discovered a method that substantially replicates in hypogonadal men natural diurnal serum testosterone patterns seen in healthy eugonadal men.

The compositions and unit dosage forms described herein can provide a daily dose of active agent. In one aspect, the daily dose is in the range of 300-700 mg and can be provided as a one or two unit dosage form option. In one aspect, the daily dose is in the 600-1200 mg range and can be provided as two, three or four unit dosage form (capsule) options: all at once (QD), or one unit dosage form in the morning and one unit dosage form in the evening (BID) or one unit dosage form during each of breakfast, during lunch, and during dinner (TID). Other examples include 3 unit dosage forms all at once (QD), or two in the morning and one in the evening (BID) or one during breakfast, one during lunch, and one during dinner (TID). These times are general references for example the doses can be taken 10 minutes after lunch, five minutes after breakfast, etc. In one aspect, the doses are administered with a meal.

Described herein are compositions having (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and their use in treating hypogonadal males. The inventors have unexpectedly found that pharmaceutical compositions having (8R,9S,10R,13S,14S,17S)-10,13 -dimethyl-3 -oxo-1,2,6,7,8,9,11,12,14,15,16,17-dodecahydrocyclopenta[a]phenanthren-17-yl tridecanoate and one or more pharmaceutically acceptable excipients can be orally administered to hypogonadal males to produce serum testosterone levels that give daily C_{avg} values that more closely approximate those of eugonadal males. Moreover, the composition can be administered in a manner that produces diurnal serum testosterone values that are substantially similar to that of eugonadal males compared to any currently available products on the market the inventors are aware of or any other product in development. Without wishing to be bound by theory, it is believed that improved serum testosterone levels that more closely approximate those of eugonadal males translates into a number of clinical benefits to the patients as compared to products that produce lower levels. Moreover, producing diurnal testosterone patterns that more closely approximate those of eugonadal males is likewise believed to translate into a number of clinical benefits for eugonadal males as compared to products that do not substantially approximate eugonadal patterns.

It has been discovered that pharmaceutical compositions (or unit dosage forms) as described herein have a unique daily dose range for which, upon daily administration to each subject in a group (e.g., of at least for example 12 hypogonadal males) for a period of at least 84 days, provides a serum testosterone C_{avg} of 300 ng/dL to 1100 ng/dL in at least 75% of the hypogonadal males in the group, and at least one of the following:
a steady state serum T concentration of <300 ng/dL for no more than 7 hours in a 24-hour period in 50% or less of the subjects.
a steady state serum T concentration of >300 ng/dL for at least 12-24 hours post-dosing in a 24-hour period in majority of the subjects.
a steady state serum T concentration serum T levels of <300 ng/dL for no more than 7 hours in a 24-hour period in 50% or less subjects, 300 ng/dL for at least 12-24 hours post-dosing in a 24-hour period in majority of the subjects.
a serum testosterone Cₘₐₓ of less than 1500 ng/dL in at least 85% of the subjects in the group;
a serum testosterone Cₘₐₓ of about 1800 ng/dL to about 2500 ng/dL in 5% or less of the subjects in the group; and
a serum testosterone Cₘₐₓ greater than 2500 ng/dL in about 1% or less of the subjects in the group.
The compositions and unit dosage forms can be prepared by any suitable method known to the skilled artisan or developed in view of the teachings herein. In one specific aspect, the carrier(s) and API are brought to or maintained at a temperature at which they are flowable (e.g., above 10° C., 2020 C., 2520 C., 3020 C., 3520 C., or 4020 C.). In one aspect, the mixture of carrier and API is a clear solution at a specified temperature (e.g., above 1020 C., 2020 C., 2520 C., 3020 C., 3520 C., or 4020 C.). In one aspect, the mixture of carrier and API is a cloudy or hazy solution at a specified temperature (e.g., below 1020 C., 2020 C., 2520 C., 3020 C., 3520 C., or 4020 C.).

In one example, the composition is prepared by weighing all of the components, except the API into a clean stainless steel container and mixed together at ambient temperature or at elevated temperatures e.g., at about 25° C. to about 30° C., at about 30° C. to about 35° C., at about 35° C. to about 40° C., at about 40° C. to about 45° C., at about 45° C. to about 45° C., or 50° C. to about 70° C., using a stirrer. The API is added and stirred into the mixture of other components until the API dissolves. A predetermined quantity of this “liquid fill material” is disposed into a capsule (for example, hard gelatin capsule) to get the required API dose per dosage unit. The capsules are allowed to cool at room temperature, banded (if required) and packaged in a HDPE bottle and tightly closed with an appropriate lid. It is noted that various capsule sizes (e.g., hard gel or soft gel) are available to the skilled artisan and allow for variations in the amount of loading of API in mg per unit dosage form. Typically, soft gel capsules for oral administration have fill volumes of less than 1.5 mL, 1.3 mL or 1.25 mL with numerous incremental fill volumes in these ranges. Similarly, hard gel capsules typically have fill volumes of less than 1.25 mL, 1.10 mL or 1 mL. Due to the nature of some hard gel capsules, the total fill volume may not be useable. There is a practical limit on the temperature at which capsules can be filled—for example temperature above 4020 C. typically melt, deform, or otherwise damage soft gel capsules typically employed in the industry. Hard gel capsules are typically less sensitive to temperature and can be filled at higher temperatures e.g., above 4020 C.

In certain embodiments, any pharmaceutical composition described herein, e.g., a can be prepared by (i) combining and heating all ingredients until a molten mixture is obtained (e.g., 50-70° C.); and (ii) encapsulating an amount of molten mixture comprising a select dose (e.g., a therapeutically effective amount or a partial dose of a therapeutically effective amount) API to obtain an oral dosage form. In certain instances, the molten mixture is spray-congealed to obtain beads. In some instances, the molten mixture is sprayed onto inert cores (e.g., sugar spheres) to obtain coated cores. In certain embodiments, such beads, cores, or similar forms are encapsulated or otherwise formulated to provide an oral dosage form. In some instances, the molten mixture is admixed, uniformly dispersed, or granulated over a carrier and compressed into a tablet dosage form. In certain embodiments, prior to compression, the molten mixture/carrier composition is further mixed with one or more pharmaceutical aid including, by way of non-limiting example, glidants, lubricants, binders, or the like. In some embodiments, the carrier is a therapeutically inert carrier such as, by way of non-limiting example, microcrystalline cellulose, starch, lactose, or the like.

In various embodiments, pharmaceutical compositions described herein are formulated as oral dosage forms. Oral dosage forms are prepared by any suitable process including one or more steps of, by way of non-limiting example, agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, solvent dehydration, sonication, spheronization, spray chilling, spray congealing, spray drying, or the like.

In some embodiments, a pharmaceutical composition described herein is formulated with a substrate to form an oral dosage form. In various embodiments, substrates useful for formulating pharmaceutical compositions described herein as oral dosage forms include or comprise, by way of non-limiting example, a powder or a multiparticulate (e.g., one or more granule, one or more pellet, one or more bead, one or more spherule, one or more beadlet, one or more microcapsule, one or more millisphere, one or more mini capsule, one or more microcapsule, one or more nanocapsule, one or more nanosphere, one or more microsphere, one or more minitablet, one or more tablet, one or more capsule, or one or more combinations thereof). In certain instances, a powder constitutes a finely divided (milled, micronized, nanosized, precipitated) form of an active ingredient or additive molecular aggregates or a compound aggregate of multiple components or a physical mixture of aggregates of an active ingredient and/or additives.

The following examples are provided to promote a more clear understanding of certain embodiments of the present invention, and are in no way meant as a limitation thereon.

### EXAMPLES

### Example 1

### Solubility in Some Pharmaceutically Acceptable Carriers/Excipients

The solubility of (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate was determined in the solvents (typical pharmaceutical carriers) listed below.

| Solvent | Solubility mg/g at ~20-23° C. |
|---|---|
| Fatty Acid-Oleic acid | 114 |
| Corn glycerides-Maisine | 77 |
| Peppermint Oil | ~200 |
| Medium chain glycerides-Capmul | 68 |
| Benzyl Benzoate | 223 |
| Benzyl Alcohol | 322 |
| Ethanol | 27 |
| Cremophor RH40 | 10 |
| Corn oil | 35 |
| Olive Oil | 38 |
| Castor Oil | 55 |

This table shows that the API described herein has solubility of greater than 5 mg/g, 20 mg/g, 50 mg/g, 75 mg/g or 100 mg/g in pharmaceutically acceptable carriers (e.g., solvents) suitable as components of oral pharmaceutical compositions. Additionally, it is believed that the API in similar carriers that are solid or semi-solid at room temperature will have similar solubility at elevated temperatures (greater than 25, 30, 35, 40, 45, 50, 60, 65, 70, 75, or 8020 C.) suitable for manufacturing oral dosage forms. Typically, there is a practical upper temperature limit that is no longer suitable for manufacturing pharmaceutical compositions. Generally, the upper limit is the temperature at which significant decomposition of the API occurs.

### Example 2

### Pharmaceutical Compositions

Shown below are various compositions suitable for oral administration as described herein. In these Examples the amount of excipient adds up to 100% (does not include the API) and the API weight percent is the final weight percent in the pharmaceutical composition.

| | Composition No. | | | | | | |
|---|---|---|---|---|---|---|---|
| Component (w/w %) | 1 | 2 | 3 | 4 | 5 | 6 | 7 |
| API | 22 | 23 | 24 | 26 | 28 | 30 | 32 |
| Excipient 1 (e.g., | 35-80 | 35-80 | 35-80 | 35-80 | 35-80 | 35-80 | 35-80 |
| liquid carrier) | | | | | | | |
| Excipient 2 | 1-40 | 1-40 | 1-40 | 1-40 | 1-40 | 1-40 | 1-40 |
| (e.g., additive) | | | | | | | |
| Excipient 3 (e.g., | 0-20 | 0-20 | 0-20 | 0-20 | 0-20 | 0-20 | 0-20 |
| hydrophilic additive) | | | | | | | |
| Excipient 4 | 0.01-3 | 0.01-3 | 0.01-3 | 0.01-3 | 0.01-3 | 0.01-3 | 0.01-3 |
| (e.g., antioxidant) | | | | | | | |
| Additional Excipients | qs | qs | qs | qs | qs | qs | qs |
| (e.g., other | | | | | | | |
| pharmaceutically | | | | | | | |
| acceptable excipients) | | | | | | | |

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid; H—(O—CH₂—CH₂)ₙ—OH where n is an integer from 3 to 900; octadecanoic acid; (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene; or a combination thereof. Excipient 3 in specific compositions is a polyoxylated hydrogenated vegetable oil. Excipient 4 in specific compositions is ascorbyl palmitate. These compositions can be filled into soft gel or hard gel capsules depending on its flowability at the temperatures useful for making these dosage forms.

### Example 3

### Pharmaceutical Compositions

Shown below are various compositions suitable for oral administration as described herein. In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component (w/w %) | 10 | 11 | 12 | 13 | 14 | 15 | 16 | 17 | 18 |
| API | 23 | 24 | 25 | 26 | 27 | 28 | 29 | 30 | 31 |
| Excipient 1 (e.g., | 40-70 | 30-70 | 40-70 | 40-70 | 40-70 | 40-70 | 40-70 | 30-70 | 40-70 |
| C14-C20 fatty acid) | | | | | | | | | |
| Excipient 2 (e.g., | 0.5-20 | | | 1-20 | | | 1-20 | | |
| glyceryl palmitostearate) | | | | | | | | | |
| Excipient 3 | | 0.5-30 | | | 5-35 | | | 10-30 | |
| Excipient 4 (e.g., | | | 0.5-15 | | | 1-12 | | | 2-11 |
| polyethylene glycol | | | | | | | | | |
| (high molecular weight)) | | | | | | | | | |
| Excipient 4 | 0.01-3 | 0.01-3 | 0.01-3 | 0.01-3 | 0.01-3 | 0.01-3 | 0.01-3 | 0.01-3 | 0.01-3 |
| (e.g., anti-oxidant | | | | | | | | | |
| (ascorbyl palmitate) | | | | | | | | | |
| Additional Excipients | qs | qs | qs | qs | qs | qs | qs | qs | qs |

The API in this examples in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. Excipient 4 in specific compositions is H—(O—CH₂—CH₂)ₙ—OH where n is an integer from 3 to 900 (e.g., PEG having an average molecular weight in the range of 2000-12000). These compositions can be filled into soft gel or hard gel capsules depending on its flowability at the temperatures useful for making these dosage forms. These compositions may include a hydrophilic additive.

### Example 3

### Pharmaceutical Compositions

Shown below are various compositions suitable for oral administration as described herein. In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component (w/w %) | 19 | 20 | 21 | 22 | 23 | 24 | 25 | 26 | 27 |
| API | 23 | 24 | 25 | 26 | 27 | 28 | 29 | 30 | 31 |
| Excipient 1 | 40-90 | 40-90 | 40-90 | 40-90 | 40-90 | 40-90 | 40-90 | 40-90 | 40-90 |
| Excipient 2 | 1-20 | | 1-20 | | 1-20 | | 1-20 | | 1-20 |
| Excipient 3 | | 1-10 | | 1-10 | | 1-10 | | 1-10 | |
| Excipient 4 | 0-25 | 0-25 | 0-25 | 0-25 | 0-25 | 0-25 | 0-25 | 0-25 | 0-25 |
| Additional Excipients | qs | qs | qs | qs | qs | qs | qs | qs | qs |

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid or a combination thereof. Excipient 3 in specific compositions is H—(O—CH₂—CH₂)ₙOH where n is an integer from 3 to 900 (e.g., PEG having an average molecular weight in the range of 2000-12000). Excipient 4 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

### Example 4

### Pharmaceutical Compositions

Shown below are various compositions suitable for oral administration as described herein. In these Examples the amount of excipient adds up to 100% (does not include the API) and the API weight percent is the final weight percent in the pharmaceutical composition.

| | Composition No. | | | | | | | | |
|---|---|---|---|---|---|---|---|---|---|
| Component (w/w %) | 28 | 29 | 30 | 31 | 32 | 33 | 34 | 35 | 36 |
| API | 23 | 24 | 25 | 26 | 27 | 28 | 29 | 30 | 31 |
| Excipient 1 | 40-90 | 45-90 | 50-90 | 55-90 | 40-90 | 45-90 | 55-90 | 50-90 | 50-90 |
| Excipient 2 | 5-15 | 1-15 | 5-15 | 1-15 | | | | | 5-15 |
| Excipient 3 | | | | | 1-10 | 1-10 | 1-10 | 1-10 | |
| Excipient 4 | 0-25 | 0-25 | 0-25 | 0-25 | 0-25 | 0-25 | 0-25 | 0-25 | 0-25 |
| Additional Excipients | qs | qs | qs | qs | qs | qs | qs | qs | qs |

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Excipient 1 in specific compositions is (9Z)-octadec-9-enoic acid. Excipient 2 in specific compositions is a combination of mono-, di-, or tri-propane-1,2,3-triol esters of octadecanoic acid and hexadecanoic acid, octadecanoic acid, or a combination thereof. Excipient 3 in specific compositions is H—(O—CH₂—CH₂)ₙ—OH where n is an integer from 3 to 900 (e.g., PEG having an average molecular weight in the range of 2000-12000). Excipient 4 in specific compositions is (1R,2S,5R)-2-isopropyl-5-methylcyclohexanol or a combination of one or more of (1R,2S,5R)-2-isopropyl-5-methylcyclohexanol, (2S,5R)-2-Isopropyl-5-methylcyclohexanone, Acetic acid [(1R,2S,5R)-2-isopropyl-5-methylcyclohexyl] ester, 1,3,3-Trimethyl-2-oxabicyclo[2,2,2]octane, and (R)-1-methyl-4-(1-methylethenyl)cyclohexene. These compositions can be filled into soft gel or hard gel capsules depending on its flowability at the temperatures useful for making these dosage forms.

### Example 5

### Pharmaceutical Compositions

Shown below are various compositions suitable for oral administration as described herein. In these Examples the amount of excipient adds up to 100% (does not include the API) and the API weight percent is the final weight percent in the pharmaceutical composition

| | Composition No. | | | | | | | | |
|---|---|---|---|---|---|---|---|---|---|
| Component(w/w %) | 37 | 38 | 39 | 40 | 41 | 42 | 43 | 44 | 45 |
| API | 23 | 24 | 25 | 26 | 27 | 28 | 29 | 30 | 31 |
| Excipient 1 | 45-80 | 45-80 | 45-80 | 45-80 | 45-80 | 45-80 | 45-80 | 45-80 | 45-80 |
| (e.g., Fatty acid) | | | | | | | | | |
| Excipient 2 | 1-15 | 1-15 | 1-15 | 1-15 | 1-15 | 1-15 | 1-15 | 1-15 | 1-15 |
| Excipient 3 | 0-10 | 0-10 | 0-10 | 0-10 | 0-10 | 0-10 | 0-10 | 0-10 | 0-10 |
| Excipient 4 | 0.1-0.3 | 0.1-0.3 | 0.1-0.3 | 0.1-0.3 | 0.1-0.3 | 0.1-0.3 | 0.1-0.3 | 0.1-0.3 | 0.1-0.3 |
| Additional Excipients | qs | qs | qs | qs | qs | qs | qs | qs | qs |

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate. Excipient 1 in specific compositions is (9Z)-octadec-9-enoic acid, hexadecanoic acid or a combination thereof. Excipient 2 in specific compositions is a combination of mono-, di-, or tri-propane-1,2,3-triol esters of octadecanoic acid and hexadecanoic acid. Excipient 3 in specific compositions polyoxylated hydrogenated castor oil (Cremophor R40). Excipient 4 in specific compositions is ascorbyl palmitate. These compositions can be filled into soft gel or hard gel capsules depending on its flowability at the temperatures useful for making these dosage forms.

### Example 6

### Pharmaceutical Composition, Formulations and Unit Dosage Forms

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate.

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.

### Composition (A)

Quantity Fill
Material per
Hard Gel Capsule

| Ingredient Name | % w/w | mg |
|---|---|---|
| API | 13%-16% | 100-120 |
| Glyceryl Monolinoleate, NF | 58%-68% | 440-490 |
| Polyoxyl 40 Hydrogenated | 0%-19% | 0-125 |
| Castor Oil, NF | | |
| Ascorbyl Palmitate, NF | 0.1%-0.3% | 0.5-2.5 |
| Polyethylene Glycol 8000, NF | 3%-9% | 35-55 |
| Total | 100.0 | 733.3 |

### Composition (B)

Quantity Fill
Material per
Hard Shell Capsule

| Ingredient Name | % w/w | mg |
|---|---|---|
| API | 23%-28% | 160-200 |
| Oleic Acid, NF | 60%-70% | 450-530 |
| ((9Z)-Octadec-9-enoic acid) | | |
| Polyoxyl 40 Hydrogenated Castor Oil, | 0%-7% | 0-37 |
| NF | | |
| Ascorbyl Palmitate, NF | 0.1%-0.3% | 0.5-2.5 |
| Polyethylene Glycol 8000, NF | 3%-9% | 35-55 |
| Total | 100.0 | 750.0 |

### Composition (C)

| | | | Quantity Fill |
|---|---|---|---|
| | Quantity Fill | | Material |
| | Material per Hard | | per Softgel |
| | Gel Capsule | | Capsule |
| Ingredient Name | % w/w | mg | mg |
| API | 23%-28% | 160-205 | 304-390 |
| Oleic Acid, NF | 37%-46% | 280-330 | 532-627 |
| ((9Z)-Octadec-9-enoic acid) | | | |
| Peppermint Oil, NF | 15-21 | 120-150 | 228-285 |
| Polyoxyl 40 Hydrogenated Castor | 0-7% | 0-35 | 0-67 |
| Oil, NF | | | |
| Ascorbyl Palmitate, NF | 0.20 | 0.5-2.5 | 1.0-4.8 |
| Glyceryl Palmitostearate (Glyceryl | 9%-15% | 80-100 | 152-190 |
| Distearate, NF) | | | |
| Total | 100.0 | 750.0 | 1250 |

### Composition (D)

| | | | Quantity Fill |
|---|---|---|---|
| | Quantity Fill | | Material |
| | Material per Hard | | Softgel |
| | Gel Capsule | | Capsule |
| Ingredient Name | % w/w | mg | mg |
| API | 25%-32% | 160-205 | 304-390 |
| Oleic Acid, NF | 50%-60% | 340-400 | 646-760 |
| ((9Z)-Octadec-9-enoic acid) | | | |
| Polyoxyl 40 Hydrogenated Castor | 0%-7% | 21-32 | 0-61 |
| Oil, NF | | | |
| Stearic Acid, NF | 0%-7% | 0-32 | 0-61 |
| Glyceryl Palmitostearate (Glyceryl | 3%-13% | 47-58 | 89-110 |
| Distearate, NF; Precirol ATO 5) | | | |
| Ascorbyl Palmitate, NF | 0.1%-3% | 0.5-2.5 | 1.0-4.8 |
| Total | 100.00 | 654.60 | 1250 |

### Composition (E)

Quantity Fill
Material per
Hard Gel Capsule

| Ingredient Name | % w/w | mg |
|---|---|---|
| API | 27%-33% | 160-205 |
| Oleic Acid, NF | 50%-70% | 335-395 |
| ((9Z)-Octadec-9-enoic acid) | | |
| Polyoxyl 40 Hydrogenated Castor Oil, | 0%-7% | 0-30 |
| NF | | |
| Ascorbyl Palmitate, NF | 0.1%-0.3% | 0.5-2.5 |
| Polyethylene Glycol 8000, NF | 3%-9% | 32-42 |
| Total | 100.0 | 611 |

### Example 7

### Pharmaceutical Composition, Formulations and Unit Dosage Forms

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate.

These compositions can be made by any suitable method and filled into hard gel or soft gel capsules as appropriate. For example, the one or more of the ingredients are warmed or heated to a temperature that allows for dissolving any solid ingredients, the API is added and mixed until a homogenous mixture is obtained and the capsule can be filled at an appropriate temperature and if needed, allowed to cool to room temperature.

### Composition (F)

| | Weight Percent | Quantity |
|---|---|---|
| | of Fill | Fill Material |
| | Pharmaceutical | per Hard Gel |
| | Composition | Capsule |
| | (±1%) | (±1%) |
| Ingredient Name | % w/w | mg |
| API | 15 | 110 |
| Glyceryl | 63 | 463 |
| Monolinoleate, NF | | |
| Polyoxyl 40 | 15 | 114 |
| Hydrogenated Castor | | |
| Oil, NF | | |
| Ascorbyl Palmitate, | 0.2 | 1.5 |
| NF | | |
| Polyethylene Glycol | 6 | 44 |
| 8000, NF | | |
| Total | 100 | 733.3 |

### Composition (G)

| | Weight Percent | Quantity |
|---|---|---|
| | of Fill | Fill Material |
| | Pharmaceutical | per Hard |
| | Composition | Gel Capsule |
| | (±1%) | (±1%) |
| Ingredient Name | % w/w | mg |
| API | 24 | 183 |
| Oleic Acid, NF | 65 | 490 |
| ((9Z)-Octadec-9-enoic | | |
| acid) | | |
| Polyoxyl 40 Hydrogenated | 4 | 30 |
| Castor Oil, NF | | |
| Ascorbyl Palmitate, NF | 0.2 | 1.5 |
| Polyethylene Glycol 8000, | 6 | 45 |
| NF | | |
| Total | 100 | 750 |

### Composition (H)

| | Weight Percent | Quantity Fill | Quantity Fill |
|---|---|---|---|
| | of Fill | Material per | Material per |
| | Pharmaceutical | Hard Gel | Softgel |
| | Composition | Capsule | Capsule |
| | (±1%) | (±1%) | (±1%) |
| Ingredient Name | % w/w | mg | mg |
| API | 24 | 183 | 300 |
| Oleic Acid, NF | 41 | 308 | 513 |
| ((9Z)-Octadec-9-enoic | | | |
| acid) | | | |
| Peppermint Oil, NF | 18 | 136 | 225 |
| Polyoxyl 40 Hydrogenated | 4 | 30 | 50 |
| Castor Oil, NF | | | |
| Ascorbyl Palmitate, NF | 0.2 | 1.5 | 2.5 |
| Glyceryl Palmitostearate | 12 | 90 | 150 |
| (Glyceryl Distearate, NF) | | | |
| Total | 100 | 750 | 1241 |

### Composition (I)

| | Weight Percent | Quantity Fill | Quantity Fill |
|---|---|---|---|
| | of Fill | Material per | Material per |
| | Pharmaceutical | Hard Gel | Soft Gel |
| | Composition | Capsule | Capsule |
| | (±1%) | (±1%) | (±1%) |
| Ingredient Name | % w/w | mg | mg |
| API | 28 | 183 | 350 |
| Oleic Acid, NF | 55 | 365 | 688 |
| ((9Z)-Octadec-9- | | | |
| enoic acid) | | | |
| Polyoxyl 40 | 4 | 26 | 50 |
| Hydrogenated Castor | | | |
| Oil, NF | | | |
| Stearic Acid, NF | 4 | 26 | 50 |
| Glyceryl | 8 | 52 | 100 |
| Palmitostearate | | | |
| (Glyceryl Distearate, | | | |
| NF; Precirol ATO 5) | | | |
| Ascorbyl Palmitate, | 0.2 | 1.3 | 2.5 |
| NF | | | |
| Total | 100 | 654 | 1241 |

### Composition (J)

| | Weight Percent of | Quantity Fill |
|---|---|---|
| | Fill Pharmaceutical | Material per |
| | Composition | Hard Gel |
| | (±1%) | Capsule (±1%) |
| Ingredient Name | % w/w | mg |
| API | 30 | 183 |
| Oleic Acid, NF | 59 | 365 |
| ((9Z)-Octadec-9- | | |
| enoic acid) | | |
| Polyoxyl 40 | 4 | 24 |
| Hydrogenated | | |
| Castor Oil, NF | | |
| Ascorbyl Palmitate, | 0.2 | 1.2 |
| NF | | |
| Polyethylene | 6 | 36 |
| Glycol 8000, NF | | |
| Total | 100 | 611 |

Based on the description provided herein and the results of the clinical trial, it is now possible to provide pharmaceutical compositions similar to those described as Compositions (A)-(J) having API ((8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate) in an amount of e.g., 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, 475 mg to 500 mg, 525 mg to 550 mg, 550 mg to 575 mg, or575 mg to 600 mg. Similar composition to Compositions (A)-(J) can also have for example:
(a) a different fatty acid, an additional fatty acid or a both,
(b) a different hydrophilic surfactant, an additional hydrophilic surfactant or both,
(c) a mono- or di-glyceride in place of the fatty acid or in combination with the fatty acid,
(d) a different solidifying agent, an additional solidifying agent, or both,
(e) a different diglyceride than glyceryl palmitostearate, an additional diglyceride or both,
(f) a different antioxidant, an additional antioxidant or both,
(g) have additional additives,
(h) use menthol or another alcohol in place of or in addition to peppermint oil,
(i) use a tocopherol in place of fatty acid, in combination with fatty acid, in place of peppermint oil, in addition to peppermint oil or a combination thereof,
(j) use a different monoglyceride than glyceryl monolinoleate, an additional monoglyceride, a diglyceride in place of glyceryl monolinoleate, a diglyceride in combination with glyceryl monolinoleate or a combination thereof, or
(k) a combination of any of the above.

### Example 8

### Release Profile

The compositions, dosage forms described herein containing API can subjected to in vitro dissolution (release) testing using USP type 2 apparatus in about 1000 mL aqueous medium. The composition (e.g., dosage form) is subjected to in vitro dissolution testing using USP type 2 apparatus in about e.g., 1000 mL 8% Triton X100 solution in water at a specific temperature (e.g., 3720 C.) at 100 rpm for a specific time (e.g., 1, 2, 3, 4, 5, 10, 15, 30, 45, 60, 75, 90, 120, 180, or 240 minute time point where a sample is withdrawn and analyzed for API content (e.g., via HPLC)).

### Example 9

### Release Profile Stability

The compositions, dosage forms described herein containing API can subjected to in vitro dissolution (release) testing using USP type 2 apparatus in about 1000 mL aqueous medium as described in the above example after storage for particular amounts of time under specific conditions. FIG. 1 shows the release profile stability of composition (I) composition (e.g., unit dosage form of composition (I) in Example 7) described herein. The diamonds with solid line labeled 1 represents time point 0; the diamond with dotted line represents 1 month storage at 25° C. and 60% relative humidity (labeled 2); the square with long dashed line represents 1 month storage at 4020 C. and 75% relative humidity (labeled 3); the square with dash dot line represents 3 month storage at 2520 C. and 60% relative humidity (labeled 4); and the square with lighter solid line represents 3 month storage at 4020 C. and 75% relative humidity (labeled 5). The X-axis represents time in hours with measurements made at 15 min, 30 min, 45 min, 1 hour, 2 hours and 4 hours. The Y-axis represents percent API released in 1000 mL 8% Triton X-100 media at 37° C. with a USP Type 2 Apparatus at 100 RPM.

FIG. 2 shows the release profile stability for composition (H) at time 0 (1), 1 month stored at either 2520 C. 60% RH (2) or 4020 C. 75% RH (3), 2 months stored at either at either 2520 C. 60% RH (4) or 4020 C. 75% RH (5), and 3 months stored at either at either 2520 C. 60% RH (6) or 4020 C. 75% RH (7). RH is relative humidity. The X-axis represents time in hours with measurements made at 15 min, 30 min, 45 min, 1 hour, 2 hours, 3 hours and 4 hours. The Y-axis represents percent API released in 1000 mL 8% Triton X-100 media at 3720 C. with a USP Type 2 Apparatus at 100 RPM.

### Example 10

### Stability of API in Compositions Described Herein

The tables below represents the results from a stability study of the indicated compositions at the indicated times and conditions. The results were obtained from HPLC analysis of the samples.

| Composition | | T = 1 month | | T = 3 months | |
|---|---|---|---|---|---|
| (I) | T = 0 | 25° C./60% | 40° C./75% | 25° C./60% | 40° C./75% |
| RRT | Ambient | RH | RH | RH | RH |
| Assay (API) | 102.7% | 99.7% | 99.2% | 102.7% | 99.4% |
| Testosterone | ND | <0.05% | 0.34% | 0.09% | 1.14% |
| 0.39 | ND | ND | ND | ND | ND |
| 0.46 | ND | ND | 0.07 | 0.06 | 0.09 |
| 0.88 | 0.06 | 0.06 | 0.05 | 0.06 | 0.05 |
| 1.36 | ND | 0.05 | ND | ND | 0.05 |
| 2.52 | NR | NR | ND | ND | 0.3 |
| Total | 0.06% | 0.11% | 0.12% | 0.12% | 0.49% |
| Unspecified | | | | | |

ND = None Detected at expected retention times above 0.05%
NR = Peaks Not observed or Reported

| | | T = 1 month | | T = 3 months | |
|---|---|---|---|---|---|
| Composition | T = 0 | 25° C./60% | 40° C./75% | | 40° C./75% |
| (H) | Ambient | RH | RH | 25° C./60% RH | RH |
| Assay (API) | 102.7% | 99.7% | 99.2% | 98.8% | 97.4% |

Known Impurity

| | | | | | |
|---|---|---|---|---|---|
| Testosterone | ND | <0.05% | 0.34% | 0.10% | 0.98% |

Unspecified RC - RRT

| | | | | | |
|---|---|---|---|---|---|
| 0.39 | ND | ND | ND | ND | ND |
| 0.46 | ND | ND | 0.07 | ND | ND |
| 0.88 | 0.06 | 0.06 | 0.05 | 0.06 | 0.05 |
| 1.36 | ND | 0.05 | ND | 0.05 | 0.05 |
| 2.43 | NR | NR | NR | NR | 0.07 |
| 2.52 | NR | NR | NR | ND | 0.12 |
| Total | 0.06% | 0.11% | 0.12% | 0.10% | 0.27% |
| Unspecified | | | | | |

ND = None Detected at expected retention times above 0.05%
NR = Peaks Not observed or Reported

RRT stands for relative retention time compared to API when analyzed by HPLC. Exemplary HPLC conditions are a C18 column (5 μm), 150×3.9 mm, and 90% methano1:10% deionized water at a flow rate of 1.0 mL/min with the column at 2520 C.

### Example 11

### Pharmacokinetic Study

This example describes an open label, dose escalating single dose study of 3 periods; Period 1, 2 and 3.

Period 1: Single dose of 330 mg of API

Period 2: Single dose of 550 mg API

Period 3: Single dose of 770 mg API

The API in this example in specific compositions is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate

Subjects

Subjects were males between 18 and 80 years of age, inclusive, with documented onset of hypogonadism prior to age 65. Recorded values of T (testosterone) <300 ng/dL can be used as an acceptable way of documenting hypogonadism.

Subjects had serum total testosterone <300 ng/dL based on two blood samples obtained between 6 and 10 AM on two separate days. Previously documented lab results could be obtained during the screening visit, within 6 weeks of Day −1 for subjects not currently on androgen replacement therapy, or following washout of androgen replacement therapy. If one of the T values is not below 300 ng/dL, at the discretion of the sponsor, the subject could be enrolled as long as the average of two T values is below 300 ng/dL.

Subjects were naïve to androgen replacement or discontinued current treatment and completed a washout of 12 weeks following intramuscular androgen injections; 4 weeks following topical or buccal androgens; 3 weeks following oral androgens, or, in the opinion of the investigator, the subject has had an adequate washout window to be eligible. Washout must be completed prior to collection of baseline serum testosterone samples to determine study eligibility.

Each dose was administered in the morning (designated 0 hours), approximately 30 minutes after a standard breakfast. There was at least three (3) day washout between Period 1, 2 and 3 (from end of a Period to the Start of next).

For Periods 1, 2 and 3, confinement began about 12 hours prior to the anticipated dosing on Day 1 and end after the 24 hour post-dose blood draw.

Period 1

Blood samples at (hours) −12, −2, 0, 2, 4, 6, 8, 12, 16, 20, and 24

Period 2

Blood samples at (hours) 0, 2, 4, 6, 8, 12, 16, 20, and 24

Period 3

Blood samples at (hours) 0, 2, 4, 6, 8, 12, 16, 20, and 24

Serum PK analyses were performed using noncompartmental methods with WinNonlin™ Professional Version 5.3 or higher (Pharsight Corp., Mountain View, Calif.) and Excel 2007 or higher (Microsoft Corp., Seattle, Wash.), respectively.

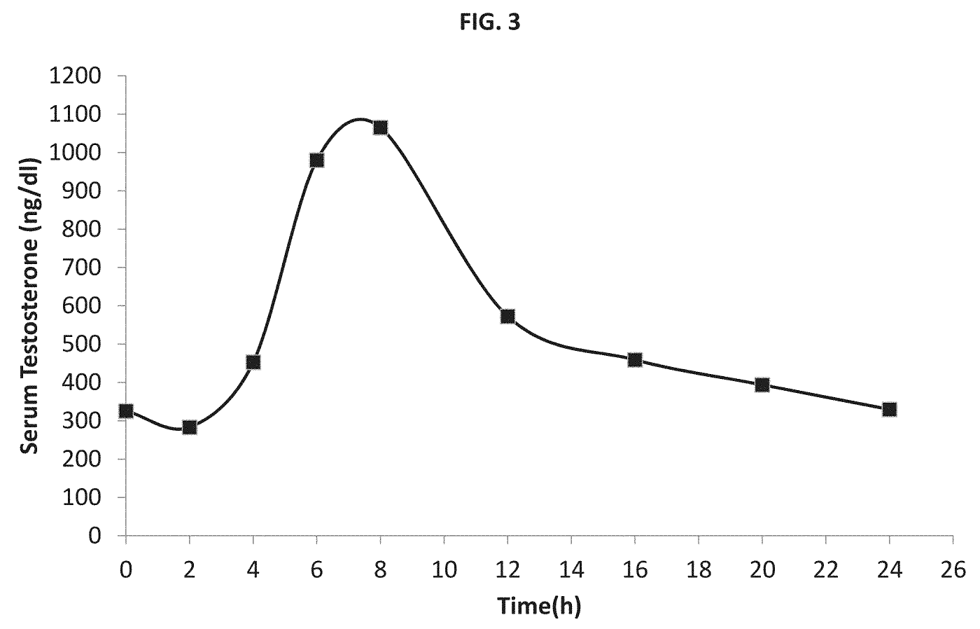

A summary of the results from this study are shown in FIG. 3 and in the Table below.

TABLE I

| | 330 mg | 550 mg | 770 mg |
|---|---|---|---|
| PK Parameter | (N = 12) | (N = 10) | (N = 10) |
| Cₘₐₓ (ng/dL) | 830 | 1108 | 1231 |
| C_{ave, 24 h} (ng/dL) | 457 | 542 | 596 |
| % Subjects with Cₘₐₓ | 0 | 0 | 0 |
| >2500 (ng/dL) | | | |
| Cₘₐₓ/Cₐᵥₑ, 24 h | 1.82 | 2.04 | 2.07 |

All subjects in this study at each dose had C_{ave,24h} (ng/dL) in the 300 to 1140 range.

A plot of a single dose (550 mg of API) pharmacokinetic study in a population of hypogonadal males (n=10) with composition (F) of Example 7 is shown in FIG. 3.

### Example 12

This example describes a randomized, open-label, single-dose, two group, three-period, five-treatment study to evaluate pharmacokinetics of the API ((8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate) formulations/unit dosage forms in healthy postmenopausal women. The study was conducted in postmenopausal women because they have very low levels of endogenous testosterone which makes the results easier to interpret and extrapolate to hypogonadal males.

Five formulations were tested (Treatments (A)-(E)). All formulations contained (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate and different excipients. The study was a single-center, open-label, single-dose, five-treatment, three-period, two-group study during which postmenopausal women were randomly assigned to one of the two groups (Group 1, Group 2) with eight (8) subjects in each group.

Subjects assigned to Group 1 received Treatments (A), (B) and (C) in a randomized cross-over way in three Periods (Period 1, 2 or 3) with a washout of at least 3 days between treatments.

Subjects assigned to Group 2 received Treatment (A), (D) and (E) in a randomized cross-over way in three Periods (Period 1, 2 or 3) with a washout of at least 3 days between treatments.

For each treatment, subjects entered the study site approximately 12 hours before anticipated dosing time and were sequestered for 36 hours. During this time, serial blood samples (8.5 mL per sample) were collected and safety variables assessed over the 24 hour after each study drug administration. Subjects were released from the study site after 24-hour blood collection at the end of each treatment period and returned for next treatment following a minimum of 3-day washout period. Safety and tolerability were assessed throughout the study.

All the treatments involved a total of 550 mg of testosterone tridecanoate per dose as follows:

1) Group 1 (Period 1, 2 or 3)

a) Treatment A: Composition (F) 110 mg (5 capsules each dose);

b) Treatment B: Composition (G) 183.3 mg (3 capsules each dose);

c) Treatment C: Composition (H) 183.3 mg (3 capsules each dose);

2) Group 2 (Period 1, 2 or 3)

a) Treatment A: Composition (F) 110 mg (5 capsules each dose);

b) Treatment D: Composition (I) 183.3 mg (3 capsules each dose)

c) Treatment E: Composition (J) 183.3 mg (3 capsules each dose)

Each dose was administered in the morning (designated 0 hours) approximately 30 minutes after a standard breakfast. Venous blood samples were collected as follows: 0 (pre-AM dose), 2, 3, 4, 5, 6, 8, 12, 16, 20 and 24 hours following administration of each treatment.

Serum PK analyses were performed using noncompartmental methods with WinNonlin™ Professional Version 5.3 or higher (Pharsight Corp., Mountain View, Calif.) and Excel 2007 or higher (Microsoft Corp., Seattle, Wash.), respectively.

The results of these studies showed that the treatments were bioequivalent (e.g., bioavailability (AUC_{0-∞}) and Cₘₐₓ within 80% to 125%) to treatment (A). Thus, the composition described herein (carrier+(additive and/or stabilizing agent)) provide advantageous loading of API with the desirable properties discussed herein. This study shows that due to the higher loading API, the number of unit dosage forms can be reduced.

### Example 13

### Dissolution/Release vs. Bioavailability

A clinical trial in humans was conducted with compositions made from or comprising an ester of (8R,9S,10R,13S,14S,17S)-17-hydroxy-10,13-dimethyl-1,2,6,7,8,9,11,12,14,15,16,17-dodecahydrocyclopenta[a]phenanthren-3-one. Single dose pharmacokinetic parameters were determined for the compositions in which the same mg amount of testosterone ester was dosed but the unit dosage forms had different dissolution/release parameters. Thirty (30) minutes before administration of each study formulation, subjects were served the following standardized high-fat, high calorie breakfast, as recommended in the U.S. Food and Drug Administration (FDA) guidance document “Food-Effect Bioavailability and Bioequivalence Studies. At least 10 subjects were in each group. The subjects were healthy post-menopausal females 45 years of age or greater to minimize effects related to endogenous ester of (8R,9S,10R,13S,14S,17S)-17-hydroxy-10,13-dimethyl-1,2,6,7,8,9,11,12,14,15,16,17-dodecahydrocyclopenta[a]phenanthren-3-one.

Ester of (8R,9S,10R,13S,14S,17S)-17-hydroxy-10,13-dimethyl-1,2,6,7,8,9,11,12,14,15,16,17-dodecahydrocyclopenta[a]phenanthren-3-one Unit Dosage Form A set to normal:

AUC₀₋ₜ=1

AUC_{0-∞}=1

Ester of (8R,9S,10R,13S,14S,17S)-17-hydroxy-10,13-dimethyl-1,2,6,7,8,9,11,12,14,15,16,17-dodecahydrocyclopenta[a]phenanthren-3-one Unit Dosage Form B:

AUC₀₋ₜ normalized to Dosage Form A (B/A)=0.73

AUC_{0-∞} normalized to Dosage Form A (B/A)=0.73

Ester of (8R,9S,10R,13S,14S,17S)-17-hydroxy-10,13-dimethyl-1,2,6,7,8,9,11,12,14,15,16,17-dodecahydrocyclopenta[a]phenanthren-3-one Unit Dosage Form C:

AUC₀₋ₜ normalized to Dosage Form A (C/A)=0.46

AUC_{0-∞} normalized to Dosage Form A (C/A)=0.46 ester of (8R,9S,10R,13S,14S,17S)-17-hydroxy-10,13-dimethyl-1,2,6,7,8,9,11,12,14,15,16,17-dodecahydrocyclopenta[a]phenanthren-3-one Unit Dosage Form D:

AUC₀₋ₜ normalized to Dosage Form A (D/A)=0.40

AUC_{0-∞} normalized to Dosage Form A (D/A)=0.40

Ester Formulation In Vitro Release Profiles

| | Time to | | Time to | |
|---|---|---|---|---|
| Formulation | Release 50% | | Release 90% | |
| Ester Unit Dosage Form A | 0.5 | hours | 1 | hour |
| Ester Unit Dosage Form B | 1 | hour | 3 | hours |
| Ester Unit Dosage Form C | 2 | hours | 5 | hours |
| Ester Unit Dosage Form D | 5.5 | hours | 11 | hour |

Unit dosage form A and B are similar to those described herein whereas unit dosage forms C and D have incorporate control releases (e.g., 15%+HPMC (e.g., hypromellose 100 cP (1(100) or 4000 cP (K4M)) agents and other excipients at levels where they substantially retard release.

As can be seen from this study, the bioavailability of the formulation decreases as the in vitro release time increases.

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

What is claimed is:

1. An oral pharmaceutical composition comprising: (a) a (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate or (8R,9S,10R,13S,14S,17S)-10,13-dimethyl-3-oxo-1,2,6,7,8,9,11,12,14,15,16,17-dodecahydrocyclopenta[a]phenanthren-17-yl tetradecanoate active pharmaceutical ingredient (API) in an amount ranging from 10-50% w/w in a liquid pharmaceutical carrier wherein the solubility of the API is greater than 5 mg/g and (b) one or more additives or stabilizing agents that allow for loading of API at levels greater than the solubility of the API in the pharmaceutical carrier without substantially compromising biovailability, release profile or release profile stability and (c) optionally one or more pharmaceutically acceptable excipients.

2. The oral pharmaceutical composition of claim 1 wherein the API is present in an amount ranging from 23-32% w/w.

3. The oral pharmaceutical composition of claim 2, wherein the API is (8R,9S,10R,13S,14S,17S)-10,13-dimethyl-3-oxo-1,2,6,7,8,9,11,12,14,15,16,17-dodecahydrocyclopenta[a]phenanthren-17-yl tridecanoate.

4. The oral pharmaceutical composition of claim 1 wherein the liquid pharmaceutically acceptable carrier is present in an amount ranging from 30% to 70% w/w.

5. The oral pharmaceutical composition of claim 1 wherein the additive or stabilizing agent is present in an amount ranging from 1-30% w/w.

6. The oral pharmaceutical composition of claim 1 said composition comprising octadecanoic acid, (9Z)-octadec-9-enoic acid, hexadecanoic acid, 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1, 2, 3-trihexadecanoyl-glycerol, 1, 2, 3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol, or 1,3-dihexadecanoyl-2-octadecanoyl-glycerol, or a combination thereof.

7. The oral pharmaceutical composition of claim 1 said composition comprising octadecanoic acid, (9Z)-octadec-9-enoic acid, hexadecanoic acid, 1-octadecanoyl-2-hexadecanoyl-glycerol, 1-octadecanoyl-3-hexadecanoyl-glycerol, 1-hexadecanoyl-2-octadecanoyl-glycerol, 1,2-dioctadecanoyl-glycerol, 1,3-dioctadecanoyl-glycerol, 1,2-dihexadecanoyl-glycerol, 1,3-dihexadecanoyl-glycerol, 1,2,3-trihexadecanoyl-glycerol, 1,2,3-trioctadecanoyl-glycerol, 1,2-dioctadecanoyl-3-hexadecanoyl-glycerol, 1,3-dioctadecanoyl-2-hexadecanoyl-glycerol, 1,2-dihexadecanoyl-3-octadecanoyl-glycerol, 1,3-dihexadecanoyl-2-octadecanoyl-glycerol, or a combination thereof present in an amount ranging from about 30% to about 75%.

8. The oral pharmaceutical composition of claim 1 which is a non-liquid at room temperature.

9. The oral pharmaceutical composition of claim 1 which is a non-solid at room temperature.

10. The oral pharmaceutical composition of claim 1 formulated as a hard gel capsule or a soft gel capsule.

11. The oral pharmaceutical composition of claim 1 which when administered as 1, 2, 3, 4, 5, or 6 unit dosage forms per day to a hypogonadal male provides C_{avg} serum testosterone levels of greater than 300 ng/dL.

12. The oral pharmaceutical composition of claim 1 further comprising a compound of formula: H—(O—CH₂—CH₂)ₙ—OH where n is an integer from 5 to 2000.

13. The oral pharmaceutical composition of claim 1 further comprising 0.1% to 20% of a compound of formula: H—(O—CH₂—CH₂)ₙ—OH where n is an integer from 9 to 500.

14. The oral pharmaceutical composition of claim 1 further comprising 1% to 10% of a compound of formula: H—(O—CH₂—CH₂)ₙ—OH where n is an integer from 10 to 300.

15. The oral pharmaceutical composition of claim 1 further comprising 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof.

16. The oral pharmaceutical composition of claim 1 further comprising 2-Isopropyl-5-methylcyclohexanone; (2S,5R)-2-Isopropyl-5-methylcyclohexanone; acetic acid [(1R,2S,5R)-2-isopropyl-5-methylcyclohexyl] ester; acetic acid [(2-isopropyl-5-methylcyclohexyl] ester; (1R,2S,5R)-2-isopropyl-5-methylcyclohexanol; (2-isopropyl-5-methylcyclohexanol; or a combination thereof which is present in an amount ranging from 5% to about 40% (w/w).

17. The oral pharmaceutical composition of claim 1 further comprising a polyoxyethylated oil; a polyoxyethylated hydrogenated vegetable oil; a polyoxyethylated hydrogenated oil; a polyoxyethylated hydrogenated castor oil; or a combination thereof.

18. The oral pharmaceutical composition of claim 1 further comprising a polyoxyethylated oil; a polyoxyethylated hydrogenated vegetable oil; a polyoxyethylated hydrogenated oil; a polyoxyethylated hydrogenated castor oil; or a combination thereof, in an amount ranging from 0% to about 25% w/w.

19. The oral pharmaceutical composition of claim 1 wherein the API is present in an amount ranging from 150 mg to about 400 mg per unit dosage form.

20. The oral pharmaceutical composition of claim 1 which is a unit dosage form that when tested with a USP type 2 paddle apparatus having 1000 mL of 8% octoxynol-9 (Triton-100) in water at 3720 C. (±0.5) (a) releases 80% or more of the active pharmaceutical ingredient at 4, 3, 2, 1, 0.5, or 0.25 hours (b) releases less than 100% at 4, 3, 2, 1, 0.5, or 0.25 hours (c) releases about 100% at 5, 4, 3, 2, 1, 0.5, or 0.25 hours or (d) a combination of one, two, or three of (a)-(c).
